(19) United States
(12) Patent Application Publication
(10) Pub. No.: US 2025/0147035 A1
(43) Pub. Date: May 8, 2025

(54) METHODS FOR DETECTION OF POST-TRANSLATIONAL MODIFICATIONS

(71) Applicant: Quantum-Si Incorporated, Branford, CT (US)

(72) Inventor: Brian Reed, Madison, CT (US)

(73) Assignee: Quantum-Si Incorporated, Branford, CT (US)

(21) Appl. No.: 18/939,364

(22) Filed: Nov. 6, 2024

Related U.S. Application Data
(60) Provisional application No. 63/596,612, filed on Nov. 6, 2023.

(51) Int. Cl. G01N 33/68

(52) U.S. Cl. CPC G01N 33/6818; G01N 2440/14

(57) ABSTRACT

The disclosure provides methods and compositions that enable the identification of polypeptides having one or more post-translational modifications. In some embodiments, the disclosure provides a method (e.g., a single-molecule measurement method) comprising contacting a single polypeptide with one or more post-translational modification-specific (PTM-specific) affinity reagents; and identifying whether the single polypeptide comprises a post-translational modification (PTM) by determining a luminescence signature representative of the binding interaction(s) between the single polypeptide and the one or more PTM-specific affinity reagents.

### CROSS-REFERENCE TO RELATED APPLICATIONS

This application claims priority under 35 U.S.C. § 119(e) to U.S. Provisional Patent Application No. 63/596,612, filed Nov. 6, 2023, which is hereby incorporated by reference in its entirety.

### BACKGROUND

Proteins represent the fundamental building blocks of life, driving key biological and cellular processes. Protein function is driven by its structure, including its sequence. In adjacent fields, like genomics, advances in sequencing technology have proven extremely valuable in improving our understanding of the progression of complex human disease.

Post-translational modifications (e.g., phosphorylation) impact protein function and structure. Identification of polypeptides having post-translational modifications and determining the specific location of a post-translational modification within a polypeptide has historically been challenging, as methodologies have been generally limited to ensemble-based methods.

### SUMMARY

The inventors of the disclosure have identified a novel methodology for the identification of post-translational modifications (PTMs) within one or more polypeptides. Specifically, the inventors have identified a methodology for identifying PTMs in a single molecule context (and not merely in an ensemble context). These methods enable the determination of precise locations of PTMs within a single polypeptide and single-molecule level determinations of proteoform distributions within a sample.

Accordingly, in some aspects, the disclosure provides a single-molecule measurement method comprising (a) contacting a single polypeptide with one or more post-translational modification-specific (PTM-specific) affinity reagents; and (b) identifying whether the single polypeptide comprises a post-translational modification (PTM) by determining a luminescence signature representative of the binding interaction(s) between the single polypeptide and the one or more PTM-specific affinity reagents.

In some embodiments, the method further comprises (c) contacting the single polypeptide with one or more terminal amino acid recognition molecules; and (d) detecting a series of signal pulses indicative of association of the one or more terminal amino acid recognition molecules with successive amino acids exposed at a terminus of the single polypeptide while the single polypeptide is being degraded. In some embodiments, the single polypeptide is a peptide (e.g., a peptide fragment of a full-length protein).

In some embodiments, the single polypeptide is a protein (e.g., a full-length protein). In some embodiments, the method further comprises: contacting the full-length protein with one or more protein-specific affinity reagents, wherein each protein-specific affinity reagent binds to an amino acid that does not comprise a PTM; and determining a further luminescence signature representative of the binding interaction(s) between the single polypeptide and the one or more protein-specific affinity reagents, wherein the further luminescence signature is further indicative of whether the single polypeptide comprises the PTM. In some embodiments, the one or more protein-specific affinity reagents comprise antibodies or aptamers. In some embodiments, the method further comprises: (c) fragmenting the full-length protein into a plurality of peptides; (d) contacting one or more peptides of the plurality with one or more terminal amino acid recognition molecules; and (e) detecting a series of signal pulses indicative of association of the one or more terminal amino acid recognition molecules with successive amino acids exposed at each terminus of the one or more peptides while the one or more peptides are being degraded.

Further aspects of the disclosure provide a method of polypeptide sequencing comprising: (a) contacting an array (e.g., a chip array) comprising a plurality of compartments with a plurality of polypeptides; (b) immobilizing each polypeptide of the plurality of polypeptides to a surface of the array (e.g., chip array); (c) contacting the plurality of polypeptides with one or more post-translational modification-specific (PTM-specific) affinity reagents; and (d) identifying whether each polypeptide comprises a post-translational modification (PTM) by determining the luminescence signature representative of the binding interaction(s) between each polypeptide and the one or more PTM-specific affinity reagents.

In some embodiments, the method further comprises: (e) contacting each polypeptide with one or more terminal amino acid recognition molecules; and (f) detecting a series of signal pulses indicative of association of the one or more terminal amino acid recognition molecules with successive amino acids exposed at a terminus of each polypeptide while each polypeptide is being degraded, thereby sequencing each polypeptide.

In some embodiments, the plurality of polypeptides are proteins (e.g., full-length proteins). In some embodiments, the method further comprises: (e) fragmenting the full-length proteins into a plurality of peptides; (f) contacting one or more peptides of the plurality with one or more terminal amino acid recognition molecules; and (g) detecting a series of signal pulses indicative of association of the one or more terminal amino acid recognition molecules with successive amino acids exposed at each terminus of the one or more peptides while the one or more peptides are being degraded, thereby sequencing the one or more peptides.

Further aspects of the disclosure provide a method of characterizing proteoforms of a polypeptide comprising: (a) contacting an array (e.g., a chip array) comprising a plurality of compartments with a sample comprising a first proteoform of a polypeptide and a second proteoform of a polypeptide, wherein the post-translational modification (PTM) profile of the first proteoform is different than the PTM profile of the second proteoform; (b) immobilizing the first proteoform to a surface of a first compartment of the array (e.g., chip array) and the second proteoform to a surface of a second compartment of the array (e.g., chip array); (c) contacting the first proteoform and the second proteoform with one or more post-translational modification-specific (PTM-specific) affinity reagents; and (d) identifying whether the first proteoform and/or the second proteoform comprises a post-translational modification (PTM) by determining the luminescence signature representative of the binding interaction(s) between each proteoform and the one or more PTM-specific affinity reagents.

In some embodiments, the method further comprises: (e) contacting the first proteoform and/or the second proteoform with one or more terminal amino acid recognition molecules; and (f) detecting a series of signal pulses indicative of association of the one or more terminal amino acid recognition molecules with successive amino acids exposed at a terminus of each proteoform while each proteoform is being degraded, thereby sequencing the first proteoform and the second proteoform.

In some embodiments, the first and second proteoforms are proteins (e.g., full-length proteins). In some embodiments, the method further comprises: contacting the first and second proteoforms with one or more protein-specific affinity reagents, wherein each protein-specific affinity reagent binds to an amino acid that does not comprise a PTM; and determining a further luminescence signature representative of the binding interaction(s) between the one or more protein-specific affinity reagents and the first proteoform and/or second proteoform, wherein the further luminescence signature is further indicative of whether the first proteoform and/or the second proteoform comprises a PTM. In some embodiments, the one or more protein-specific affinity reagents comprise antibodies or aptamers. In some embodiments, the method further comprises: (e) fragmenting the full-length proteins into a plurality of peptides; (f) contacting one or more peptides of the plurality with one or more terminal amino acid recognition molecules; and (g) detecting a series of signal pulses indicative of association of the one or more terminal amino acid recognition molecules with successive amino acids exposed at each terminus of the one or more peptides while the one or more peptides are being degraded, thereby sequencing the one or more peptides.

In some embodiments, the one or more PTM-specific affinity reagents are antibodies or aptamers. In some embodiments, the one or more PTM-specific affinity reagents specifically bind to an amino acid comprising a phosphorylation, a glycosylation, acetylation, ADP-ribosylation, citrullination, formylation, N-linked glycosylation, O-linked glycosylation, hydroxylation, methylation, myristoylation, neddylation, nitration, oxidation, palmitoylation, prenylation, S-nitrosylation, sulfation, sumoylation, or ubiquitination.

In some embodiments, the one or more PTM-specific affinity reagents specifically bind to phospho-tyrosine, phospho-serine, or phospho-threonine. In some embodiments, the one or more PTM-specific affinity reagents comprise one or more PTM-specific affinity reagents selected from Table 1. In some embodiments, the one or more PTM-specific affinity reagents comprise one or more PTM-specific affinity reagents selected from PM49, PS33, and PM12.

In some embodiments, the one or more PTM-specific affinity reagents is labeled. In some embodiments, the label is a luminescent label or a conductivity label. In some embodiments, the luminescent label comprises at least one fluorophore dye molecule. In some embodiments, the luminescent label comprises 20 or fewer fluorophore dye molecules.

In some embodiments, the polypeptide(s) are contacted with two or more PTM-specific affinity reagents at the same time. In some embodiments, each of the two or more PTM-specific affinity reagents comprise a unique label relative to the other PTM-specific affinity reagents.

In some embodiments, the polypeptide(s) are contacted in series with a first PTM-specific affinity reagent and a second PTM-specific affinity reagent, optionally wherein the first PTM-specific affinity reagent is removed (e.g., by washing) prior to addition of the second PTM-specific affinity reagent.

In some embodiments, determining the luminescence signature comprises detecting a series of signal pulses indicative of association of the one or more PTM-specific affinity reagents with the PTM of the polypeptide(s).

In some embodiments, detecting a series of signal pulses indicative of association of the one or more PTM-specific affinity reagents with the PTM of the polypeptide(s) allows for a determination of the type of amino acids located at positions in proximity to the PTM of the polypeptide(s).

In some embodiments, detecting a series of signal pulses indicative of association of the one or more PTM-specific affinity reagents with the PTM of the polypeptide(s) allows for a determination of the location of the PTM within the polypeptide(s).

In some embodiments, detecting a series of signal pulses indicative of association of the one or more PTM-specific affinity reagents with the PTM of the polypeptide(s) assists with a determination of the amino acid sequence of the polypeptide(s).

In some embodiments, the PTM is an amino acid comprising a phosphorylation, a glycosylation, acetylation, ADP-ribosylation, citrullination, formylation, N-linked glycosylation, O-linked glycosylation, hydroxylation, methylation, myristoylation, neddylation, nitration, oxidation, palmitoylation, prenylation, S-nitrosylation, sulfation, sumoylation, or ubiquitination. In some embodiments, the PTM is phospho-tyrosine, phospho-serine, or phospho-threonine.

In some embodiments, contacting the polypeptide(s) with one or more terminal amino acid recognition molecules further comprises contacting the polypeptide(s) with a cleaving reagent. In some embodiments, the cleaving reagent is an aminopeptidase.

In some embodiments, the method allows for identification of the presence of the PTM at any location in the polypeptide(s).

In some embodiments, the method further comprises washing the polypeptide(s) after determining the luminescence signature of the polypeptide(s) in the presence of the one or more PTM-specific affinity reagents.

In some embodiments, the method further comprises fragmenting the polypeptide(s) prior to step (a). In some embodiments, the fragmenting is done by cleaving (e.g., chemically cleaving) and/or digesting (e.g., enzymatically digesting using a peptidase) the polypeptide(s).

In some embodiments, association of the one or more terminal amino acid recognition molecules with each type of amino acid exposed at the terminus produces a characteristic pattern in the series of signal pulses that is different from other types of amino acids exposed at the terminus, optionally wherein the characteristic pattern comprises a portion of the series of signal pulses.

In some embodiments, a signal pulse of the characteristic pattern corresponds to an individual association event between a terminal amino acid recognition molecule and an amino acid exposed at the terminus. In some embodiments, the signal pulse of the characteristic pattern comprises a pulse duration that is characteristic of a dissociation rate of binding between the terminal amino acid recognition molecule and the amino acid exposed at the terminus. In some embodiments, each signal pulse of the characteristic pattern is separated from another by an interpulse duration that is characteristic of an association rate of terminal amino acid recognition molecule binding.

In some embodiments, the characteristic pattern corresponds to a series of reversible terminal amino acid recognition molecule binding interactions with the amino acid exposed at the terminus of the single polypeptide molecule. In some embodiments, the characteristic pattern is indicative of the amino acid exposed at the terminus of the single polypeptide molecule and an amino acid at a contiguous position.

Further aspects of the disclosure provide a composition comprising three or more PTM-specific affinity reagents.

In some embodiments, the three or more PTM-specific affinity reagents are antibodies or aptamers. In some embodiments, the three or more PTM-specific affinity reagents specifically bind to an amino acid comprising a phosphorylation, a glycosylation, acetylation, ADP-ribosylation, citrullination, formylation, N-linked glycosylation, O-linked glycosylation, hydroxylation, methylation, myristoylation, neddylation, nitration, oxidation, palmitoylation, prenylation, S-nitrosylation, sulfation, sumoylation, or ubiquitination.

In some embodiments, the three or more PTM-specific affinity reagents comprise at least one (e.g., 1, 2, 3, 4, 5, or more) PTM-specific affinity reagent selected from Table 1. In some embodiments, the three or more PTM-specific affinity reagents comprise PM49. In some embodiments, the three or more PTM-specific affinity reagents comprise PS33. In some embodiments, the three or more PTM-specific affinity reagents comprise PM12. In some embodiments, the three or more PTM-specific affinity reagents comprise PM49, PS33, and PM12.

In some embodiments, the composition further comprises a PTM-specific affinity reagent that specifically binds to phospho-tyrosine, a PTM-specific affinity reagent that specifically binds to phospho-serine, and a PTM-specific affinity reagent that specifically binds to phospho-threonine.

In some embodiments, the one or more PTM-specific affinity reagents is labeled. In some embodiments, the label is a luminescent label or a conductivity label. In some embodiments, the luminescent label comprises at least one fluorophore dye molecule. In some embodiments, the luminescent label comprises 20 or fewer fluorophore dye molecules.

In some embodiments, each of the three or more PTM-specific affinity reagents comprise a unique label relative to the other PTM-specific affinity reagents.

In some embodiments, the composition further comprises a sample comprising polypeptides. In some embodiments, the composition further comprises a cleaving reagent (e.g., an aminopeptidase).

In some aspects, the disclosure provides a method of polypeptide analysis comprising: (a) contacting a polypeptide with a composition comprising one or more PTM-specific affinity reagents; (b) monitoring a signal for a first series of signal pulses corresponding to binding interactions between the one or more PTM-specific affinity reagents and the polypeptide; (c) contacting the polypeptide with a composition comprising one or more amino acid recognition molecules; (d) monitoring the signal for a second series of signal pulses corresponding to binding interactions between the one or more amino acid recognition molecules and the polypeptide; and (e) determining one or more chemical characteristics of the polypeptide based on one or more characteristic patterns in the first and/or second series of signal pulses.

In some embodiments, (e) comprises identifying at least one PTM in the polypeptide based on a characteristic pattern in the first series of signal pulses. In some embodiments, the at least one PTM comprises phospho-threonine, phospho-serine, phospho-tyrosine, or any combination thereof. In some embodiments, (e) comprises identifying one or more types of amino acids in the polypeptide based on a characteristic pattern in the second series of signal pulses.

In some embodiments, the method further comprises washing the polypeptide after (b) and prior to (c). In some embodiments, the washing comprises contacting the polypeptide with a wash solution after (b) and prior to (c).

In some embodiments, the composition of (a) does not comprise a cleaving reagent. In some embodiments, the composition of (c) comprises one or more cleaving reagents. In some embodiments, the one or more PTM-specific affinity reagents comprise antibodies or aptamers. In some embodiments, the one or more PTM-specific affinity reagents comprise antibodies. In some embodiments, the one or more amino acid recognition molecules of (c) do not comprise antibodies.

In some embodiments, the polypeptide is a protein (e.g., a full-length protein). In some embodiments, the method further comprises fragmenting the full-length protein into a plurality of peptides after (b) and prior to (c).

In some embodiments, the method further comprises repeating (a) and (b) one or more times prior to (c). In some embodiments, the repeating comprises contacting the polypeptide with one or more PTM-specific affinity reagents that are different from the one or more PTM-specific affinity reagents in the composition of (a). In some embodiments, the method further comprises washing the polypeptide before the repeating.

In some embodiments, the one or more PTM-specific affinity reagents are labeled. In some embodiments, the label is a luminescent label or a conductivity label. In some embodiments, the one or more PTM-specific affinity reagents specifically bind to an amino acid comprising a PTM described herein. In some embodiments, at least one PTM-specific affinity reagent specifically binds to phospho-tyrosine, phospho-serine, or phospho-threonine. In some embodiments, the one or more PTM-specific affinity reagents comprise one or more PTM-specific affinity reagents selected from Table 1. In some embodiments, the one or more PTM-specific affinity reagents comprise a PTM-specific affinity reagent that specifically binds to phospho-tyrosine. In some embodiments, the PTM-specific affinity reagent is PM49. In some embodiments, the PTM-specific affinity reagent that specifically binds to phospho-tyrosine comprises an SH2 domain. In some embodiments, the PTM-specific affinity reagent is PS33. In some embodiments, the one or more PTM-specific affinity reagents comprise a PTM-specific affinity reagent that specifically binds to phospho-threonine. In some embodiments, the PTM-specific affinity reagent that specifically binds to phospho-threonine comprises an FHA domain. In some embodiments, the PTM-specific affinity reagent is PM12.

In some embodiments, the polypeptide is immobilized to a surface of a substrate comprising an array of compartments. In some embodiments, the polypeptide is immobilized within a first compartment of the array. In some embodiments, a different proteoform of the polypeptide is immobilized within a second compartment of the array. In some embodiments, the method comprises performing (a) to (d) for each proteoform of the polypeptide, wherein (e) comprises identifying each proteoform of the polypeptide based on differences in the one or more chemical characteristics.

In some aspects, the disclosure provides a method of polypeptide analysis comprising: (a) contacting a substrate comprising an array of compartments with a composition comprising one or more PTM-specific affinity reagents, wherein each of at least two compartments of the array comprises a different proteoform of a polypeptide; (b) detecting a first series of signal pulses corresponding to binding interactions between the one or more PTM-specific affinity reagents and a first proteoform of the polypeptide in a first compartment; (c) detecting a second series of signal pulses corresponding to binding interactions between the one or more PTM-specific affinity reagents and a second proteoform of the polypeptide in a second compartment; and (d) identifying one or more PTMs in each of the first and second proteoforms based on one or more characteristic patterns in each of the respective first and second series of signal pulses.

In some embodiments, the method further comprises sequencing the first and second proteoforms of the polypeptide. In some embodiments, the sequencing comprises identifying one or more types of amino acids in each of the first and second proteoforms of the polypeptide. In some embodiments, the sequencing comprises: (e) contacting the substrate with a composition comprising one or more amino acid recognition molecules; and (f) detecting a series of signal pulses corresponding to binding interactions between the one or more amino acid recognition molecules and successive amino acids exposed at a terminus of each proteoform while each proteoform is being degraded, thereby sequencing the first and second proteoforms.

In some embodiments, the first and second proteoforms are full-length proteins. In some embodiments, the method further comprises: fragmenting the full-length proteins into a plurality of peptides; contacting one or more peptides of the plurality with one or more terminal amino acid recognition molecules; and detecting a series of signal pulses indicative of association of the one or more terminal amino acid recognition molecules with successive amino acids exposed at each terminus of the one or more peptides while the one or more peptides are being degraded, thereby sequencing the one or more peptides.

In some embodiments, (d) comprises determining that the first and second proteoforms are different proteoforms of the polypeptide. In some embodiments, the first proteoform of the polypeptide is immobilized to a surface of the first compartment, and the second proteoform of the polypeptide is immobilized to a surface of the second compartment.

The details of certain embodiments of the disclosure are set forth in the Detailed Description. Other features, objects, and advantages of the disclosure will be apparent from the Examples, Drawings, and Claims.

### BRIEF DESCRIPTION OF THE DRAWINGS

The accompanying Drawings, which constitute a part of this specification, illustrate several embodiments of the disclosure and together with the accompanying description, serve to explain the principles of the disclosure.

FIGS. 1A-1B provide data showing the ability of an exemplary method of the disclosure to identify the presence and location of a phospho-tyrosine (a post-translational modification) in a model polypeptide. The method involved a two-stage recognition run in which peptides were contacted with a phospho-tyrosine-specific affinity reagent (PS33) prior to peptide sequencing. Peptides with phosphorylated tyrosine show fast pulsing during the pre-sequencing stage of the recognition run (FIG. 1B), while peptides without phosphorylated tyrosine do not show any pulsing during the pre-sequencing stage (FIG. 1A).

FIG. 2 provides data showing the ability of an exemplary method of the disclosure to determine that a full-length polypeptide comprises a post-translational modification.

FIG. 3 demonstrates that the methods of the disclosure can be performed on a chip (e.g., by immobilizing polypeptide targets to surfaces of the chip) and in solution (e.g., by contacting the polypeptide target(s) with PTM-specific affinity reagents (e.g., PTM-specific nanobodies) in solution.

FIGS. 4-6 provide schematics of an example single-molecule measurement method for identification and characterization of the pattern of post-translational modifications present on a single polypeptide.

FIG. 7 provides an example method in which an affinity reagent is capable of binding a site on a polypeptide of post-translational modification independently of the modification state of the site.

FIG. 8 provides Octet binding profile results showing the affinity of an exemplary PTM-specific affinity reagent for peptides comprising phosphorylated tyrosine as compared to a peptide that does not comprise a phosphorylated tyrosine.

FIG. 9 provides a schematic of an exemplary labeled PTM-specific affinity reagent for use in methods of the disclosure.

FIGS. 10A-10D provide data of an exemplary two-stage recognition run of phospho-tyrosine identification using a phospho-tyrosine-specific affinity reagent for phospho-tyrosine identification followed by peptide sequencing. Peptides with phosphorylated tyrosine show fast pulsing during the pre-sequencing stage of the recognition run, while peptides without phosphorylated tyrosine do not show any pulsing during the pre-sequencing stage. Interpulse duration during the pre-sequencing stage can be modulated by using different concentrations of a phospho-tyrosine-specific affinity reagent.

FIGS. 11A-11B provide data showing phospho-tyrosine recognition in various peptide motifs using a phospho-tyrosine-specific affinity reagent. Peptides with phosphorylated tyrosine show fast pulsing during the pre-sequencing stage of the recognition run, while peptides without phosphorylated tyrosine do not show any pulsing during the pre-sequencing stage.

FIGS. 12A-12B provide data demonstrating recognition of phospho-tyrosine at the N-terminus, C-terminus, and middle of a peptide using a phospho-tyrosine-specific affinity reagent. Peptide with phosphorylated tyrosine, especially at N-terminus show faster pulsing than C-terminus, and middle of a peptide during the pre-sequencing stage of the recognition run, while peptides without phosphorylated tyrosine do not show any pulsing in the pre-sequencing stage.

FIGS. 13A-13K provide data demonstrating recognition of phospho-tyrosine residues on peptides spiked in the CDNF library using a phospho-tyrosine-specific affinity reagent. Peptides with phosphorylated tyrosine show fast pulsing with distinct Pulse durations (PDs) for each specific p(Y) peptide sequence, during the pre-sequencing stage of the recognition run, while peptides without phosphorylated tyrosine do not show any pulsing in the pre-sequencing stage.

FIGS. 14A-14B provide Octet binding profile data showing the affinity of an exemplary PTM-specific affinity reagent for peptides comprising phosphorylated threonine as compared to peptides that do not comprise a phosphorylated threonine.

FIG. 15 provides a schematic of an exemplary labeled PTM-specific affinity reagent for use in methods of the disclosure.

FIGS. 16A-16C provide data of an exemplary two-stage recognition run of phospho-threonine identification followed by peptide sequencing using a phospho-threonine-specific affinity reagent for phospho-threonine identification. Peptides with phosphorylated threonine show fast pulsing during the pre-sequencing stage of the recognition run, while peptides without phosphorylated threonine do not show any pulsing in the pre-sequencing stage.

FIGS. 17A-17B provide data showing phospho-threonine recognition in various peptide motifs using a phospho-threonine-specific affinity reagent. Peptides with phosphorylated threonine show fast pulsing during the pre-sequencing stage of the recognition run, while peptides without phosphorylated threonine do not show any pulsing in the pre-sequencing stage.

FIGS. 18A-18B provide data demonstrating recognition of phospho-threonine at the N-terminus, C-terminus, and middle of a peptide using a phospho-threonine-specific affinity reagent. Peptides with phosphorylated threonine show fast pulsing during the pre-sequencing stage of the recognition run, while peptides without phosphorylated threonine do not show any pulsing in the pre-sequencing stage.

FIGS. 19A-19K provide data showing recognition of phospho-threonine residues spiked in the CDNF library using a phospho-threonine-specific affinity reagent. Peptides with phosphorylated threonine show fast pulsing during the pre-sequencing stage of the recognition run, while peptides without phosphorylated threonine do not show any pulsing in the pre-sequencing stage.

FIGS. 20A-20C provide data demonstrating binding affinity variation of a PTM-specific affinity reagent (PM49) to different peptide sequences and phospho-tyrosine locations within a peptide. Bulk polarization binding assays were run with 0.5 μM PM49 with YA=YAKLDEESILKQ-FITC (control); pYA=pYAKLDEESILKQ-FITC; pYQR=pYQRFLAYPDDD-FITC; or QRFpY=QRFpYLAYPDDD-FITC.

FIGS. 21A-21E provide data from polarization binding assays using a fixed concentration of PM12 with 2 μM and FITC-labeled peptides (pTQRFLAYPDDD, pYQRFLAYPDDD, pSQRFLAYPDDD, QRFpTLAYPDDD, pTAKLDEESILKQ).

FIGS. 22A-22B provide titration curves from of PM12 from a high-throughput preparation conjugated with streptavidin (HTP) or from a large-scale prep without streptavidin (LS) to TA (TAKLDEESILKQ), pTA (pTAKLDEESILKQ-PTQRF (pTQRFLAYPDDD), QRFpT (QRFpTLAYPDDD).

### DETAILED DESCRIPTION

Aspects of the disclosure relate to compositions and methods for identification of post-translation modifications (PTMs) in one or more polypeptides. In some embodiments, the methods described herein comprise contacting a polypeptide with one or more affinity reagents (e.g., labeled affinity reagents), and determining a luminescence signature representative of binding interactions between the one or more affinity reagents and the polypeptide. In some embodiments, the luminescence signature is indicative of one or more PTMs in the polypeptide.

Compositions and methods for obtaining luminescence signatures and collecting data relating to binding interactions between an affinity reagent (e.g., a labeled reagent) and a polypeptide, including methods polypeptide sequencing, are described more fully in PCT International Publication No. WO2020102741A1, filed Nov. 15, 2019, PCT International Publication No. WO2021236983A2, filed May 20, 2021, PCT International Publication No. WO2023122769A2, filed Dec. 22, 2022, PCT International Publication No. WO2024031031A2, filed Aug. 3, 2023, and PCT International Publication No. WO2024086832A1, filed Oct. 20, 2023, each of which is incorporated by reference in its entirety.

In some aspects, the disclosure provides composition and methods for identifying one or more PTMs in a polypeptide, such as a full-length protein or a peptide (e.g., one or more peptide fragments of a full-length protein). Accordingly, the term “polypeptide” as used herein can refer to a polymeric form of amino acids of any length (e.g., at least 10, at least 20, at least 30, at least 50, at least 100, 5-500, 20-500, 100-500, 5-50, 10-100, or 250-500 amino acids in length).

In some embodiments, a polypeptide refers to a protein (e.g., a full-length protein). In some embodiments, a protein can refer to any full-length, natively folded protein, such as a naturally occurring protein that has not been artificially fragmented (e.g., digested) into smaller peptide fragments. In some embodiments, a protein comprises a polymeric form of amino acids of at least 50 amino acids in length (e.g., at least 75, at least 100, at least 150, at least 250, 50-500, 50-250, 50-100, 100-250, 200-400, or 250-500 amino acids in length). In some embodiments, a protein has a molecular weight of at least 5 kilodaltons (e.g., at least 10, at least 15, at least 25, at least 50, 10-100, 10-50, 25-100, 25-50, 50-250, or 50-100 kilodaltons in size).

In some embodiments, a polypeptide refers to a peptide, such as a peptide fragment of a full-length protein. In some embodiments, a peptide refers to a polymeric form of amino acids of any length that is shorter than the full-length protein from which it is derived. In some embodiments, a peptide comprises a polymeric form of amino acids of at least 5 amino acids in length (e.g., at least 10, at least 15, at least 20, at least 25, 5-50, 10-50, 15-40, 20-60, 20-40, or 15-60 amino acids in length). Accordingly, in some embodiments, a polypeptide can refer to a peptide fragment of a protein, and the methods described herein can further comprise fragmenting a protein to produce the peptide and one or more peptide fragments of the protein. In some embodiments, the fragmenting comprises cleaving (e.g., chemically cleaving) and/or digesting (e.g., enzymatically digesting using a peptidase, such as trypsin or proteinase K) the protein to produce the peptide fragments thereof.

In some embodiments, the methods described herein can be used to identify or characterize proteoforms of a polypeptide. As used herein and known in the art, a “proteoform” refers to a specific molecular form of a protein product arising from a specific gene. The proteoform of a polypeptide encompasses the translated amino acid sequence of the polypeptide and post-translational modifications of the polypeptide. In reference to a particular polypeptide, different proteoforms refer to the range of different structures of a protein product arising from a single gene. As the presence or concentration of a particular proteoform may increase or decrease in abnormal physiological states, protein characterization at the proteoform level has a crucial importance to fully understand biological processes. Accordingly, in some embodiments, the methods of the disclosure can be used for proteoform characterization in a biological sample (e.g., cell sample, serum sample, blood sample), which can in turn provide meaningful information for therapeutic and diagnostic purposes.

Post-translational modifications (PTMs) are modifications that occur on a protein, typically catalyzed by enzymes, after translation of the protein. A PTM generally refers to the covalent addition of a functional group to a protein, proteolytic cleavage of a protein, or degradation of one or more regions of a protein. Examples of PTMs are known in the art and include, without limitation, phosphorylation, glycosylation, acetylation, ADP-ribosylation, citrullination, formylation, N-linked glycosylation, O-linked glycosylation, hydroxylation, methylation, myristoylation, neddylation, nitration, oxidation, palmitoylation, prenylation, S-nitrosylation, sulfation, sumoylation, and ubiquitination.

Aspects of the disclosure provide methods of polypeptide analysis for identifying PTMs in a polypeptide and/or characterizing proteoforms of a polypeptide. In some embodiments, the methods comprise detecting a series of signal pulses corresponding to binding interactions between one or more PTM-specific affinity reagents and the polypeptide. In some embodiments, the detected series of signal pulses provides a luminescence signature that is representative of the binding interactions, which can be used to identify the presence, location, and/or abundance of one or more PTMs in the polypeptide. In some embodiments, a characteristic pattern in the detected series of signal pulses is indicative of the presence, location, and/or abundance of one or more PTMs in the polypeptide. In some embodiments, a plurality of different characteristic patterns can be determined from a detected series of signal pulses, where each of the different characteristic patterns is indicative of a different chemical characteristic of the polypeptide (e.g., identity, location, and/or abundance of a particular type of amino acid and/or a particular type of PTM in the polypeptide). Suitable techniques for obtaining such signal pulse information and determining characteristic patterns therein have been described more fully, for example, in PCT International Publication Nos. WO2020102741A1, WO2021236983A2, WO2023122769A2, WO2024031031A2, and WO2024086832A1, each of which is incorporated by reference in its entirety.

In some embodiments, the methods described herein can be performed in parallel on a substrate comprising an array (e.g., a chip array) that comprises a plurality of compartments. In some embodiments, an array comprises between about 10,000 and about 1,000,000 compartments. The volume of a compartment may be between about 10⁻²¹ liters and about 10⁻¹⁵ liters, in some implementations. Because the compartment has a small volume, detection of single-molecule events may be possible as only about one polypeptide may be within a compartment at any given time. Statistically, some compartments may not contain a single-molecule reaction, and some compartments may contain more than one single polypeptide molecule. However, an appreciable number of compartments may each contain a single-molecule reaction (e.g., at least 30% in some embodiments), so that single-molecule analysis can be carried out in parallel for a large number of compartments.

Accordingly, aspects of the disclosure provide methods of polypeptide analysis for characterizing proteoforms of a polypeptide. In some embodiments, the methods are carried out using a substrate comprising an array of compartments, where each of at least two compartments of the array comprises a different proteoform of a polypeptide. In some embodiments, a plurality (e.g., two or more, three or more, four or more, five or more, ten or more) of compartments of the array comprise single polypeptide molecules corresponding to different proteoforms of a polypeptide. In some embodiments, the methods comprise detecting, in each compartment of the plurality, a series of signal pulses corresponding to binding interactions between one or more PTM-specific affinity reagents and a specific proteoform in the compartment. In some embodiments, a characteristic pattern in the detected series of signal pulses is indicative of the specific proteoform in the compartment. Thus, in some embodiments, different characteristic patterns in the series of signal pulses detected in different compartments can be used to distinguish between the different proteoforms of the polypeptide.

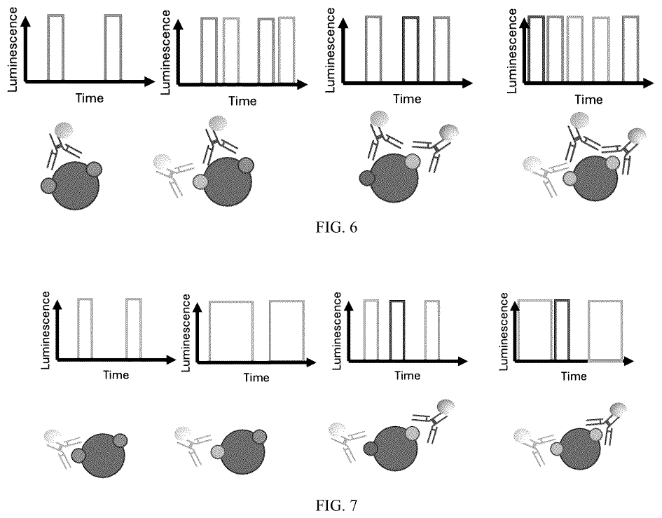

In some embodiments, the methods described herein comprise contacting a polypeptide, or different proteoforms of a polypeptide, with one or more PTM-specific affinity reagents and one or more protein-specific affinity reagents. For example, as depicted in FIG. 4, in some embodiments, different proteoforms of a polypeptide can include different PTM patterns characterized by specific sites that are modified or unmodified. In some embodiments, the different proteoforms can be contacted with one or more PTM-specific affinity reagents and one or more protein-specific affinity reagents, where each PTM-specific affinity reagent can bind a particular PTM of a polypeptide and each protein-specific affinity reagent can bind the polypeptide independently of PTMs. The different sets of affinity reagents differentially bind to different proteoforms of a polypeptide (FIG. 5), and this differential binding can be detected and evaluated to identify and/or characterize different proteoforms based on different luminescence signatures in accordance with the disclosure (FIGS. 6-7). In some embodiments, a protein-specific affinity reagent is an antibody, a nanobody, an aptamer, or an amino acid recognition molecule described herein. Techniques for producing antibodies and other such target-specific molecules that bind to a desired target are routine and well known to those skilled in the art.

### Post-Translational Modification-Specific (PTM-Specific) Affinity Reagents

A post-translational modification-specific (PTM-specific) affinity reagent is a molecule that binds to an amino acid comprising a post-translational modification (PTM). In some embodiments, the PTM-specific affinity reagent specifically binds to an amino acid comprising a PTM (e.g., binds to the amino acid having a PTM with a higher affinity than the same amino acid without the PTM).

PTM-specific affinity reagents include, for example, proteins and nucleic acids, which may be synthetic or recombinant. In some embodiments, a PTM-specific affinity reagent is an antibody (e.g., a single-chain antibody variable fragment (scFv) or VHH (Nanobody)). In some embodiments, a PTM-specific affinity reagent is an aptamer.

The PTM-specific affinity reagent can specifically bind to an amino acid comprising a phosphorylation (e.g., phospho-tyrosine, phospho-serine, or phospho-threonine), a glycosylation, acetylation (e.g., acetylated lysine), ADP-ribosylation, citrullination, formylation, (e.g., glycosylated asparagine), O-linked glycosylation (e.g., glycosylated serine, glycosylated threonine), hydroxylation, methylation (e.g., methylated lysine, methylated arginine), myristoylation (e.g., myristoylated glycine), neddylation, nitration (e.g., nitrated tyrosine), chlorination (e.g., chlorinated tyrosine), oxidation/reduction (e.g., oxidized cysteine, oxidized methionine), palmitoylation (e.g., palmitoylated cysteine), phosphorylation, prenylation (e.g., prenylated cysteine), S-nitrosylation (e.g., S-nitrosylated cysteine, S-nitrosylated methionine), sulfation, sumoylation (e.g., sumoylated lysine), or ubiquitination (e.g., ubiquitinated lysine). In some embodiments, the PTM-specific affinity reagent can specifically bind to a phospho-tyrosine, phospho-serine, or phospho-threonine amino acid.

In some embodiments, a PTM-specific affinity reagent that specifically binds to phospho-tyrosine comprises an SH2 domain. In some embodiments, a PTM-specific affinity reagent that specifically binds to phospho-tyrosine is PS33 (SEQ ID NO: 35). In some embodiments, a PTM-specific affinity reagent that specifically binds to phospho-tyrosine is PM49 (ABCAM® Biotin Anti-Phosphotyrosine antibody [EPR16871]).

In some embodiments, a PTM-specific affinity reagent that specifically binds to a phospho-threonine comprises an FHA domain. In some embodiments, a PTM-specific affinity reagent that specifically binds to phospho-threonine is PM12 (SEQ ID NO: 12).

In some embodiments, a PTM-specific affinity reagent specifically binds to a serine amino acid comprising a PTM. In some embodiments, a PTM-specific affinity reagent specifically binds to a threonine amino acid comprising a PTM. In some embodiments, a PTM-specific affinity reagent specifically binds to a tyrosine amino acid comprising a PTM. In some embodiments, a PTM-specific affinity reagent specifically binds to a lysine amino acid comprising a PTM. In some embodiments, a PTM-specific affinity reagent specifically binds to an asparagine amino acid comprising a PTM. In some embodiments, a PTM-specific affinity reagent specifically binds to an arginine amino acid comprising a PTM. In some embodiments, a PTM-specific affinity reagent specifically binds to a glycine amino acid comprising a PTM. In some embodiments, a PTM-specific affinity reagent specifically binds to a cysteine amino acid comprising a PTM. In some embodiments, a PTM-specific affinity reagent specifically binds to a methionine amino acid comprising a PTM.

In some embodiments, a PTM specific affinity reagent comprises an amino acid sequence that is at least 80% (e.g., at least 80%, at least 85%, at least 90%, at least 95%, or at least 99%) identical to any one of SEQ ID NOs: 1-35. In some embodiments, a PTM-specific affinity reagent comprises the amino acid sequence of any one of SEQ ID NOs: 1-35. In some embodiments, a PTM-specific affinity reagent consists of the amino acid sequence of any one of SEQ ID NO: 1-35.

TABLE 1
PTM-specific affinity reagent amino acid sequences.

| | | | SEQ |
|---|---|---|---|
| | | | ID |
| Description | Source | Sequence | NO |
| PM01 | Human/14-3-3 | MDDREDLVYQAKLAEQAERYDEMVESMKKVAGMDVELTV | 1 |
| | protein epsilon | EERNLLSVAYKNVIGARRASWRIISSIEQKEENKGGEDKL | |
| | | KMIREYRQMVETELKLICCDILDVLDKHLIPAANTGESKVF | |
| | | YYKMKGDYHRYLAEFATGNDRKEAAENSLVAYKAASDIA | |
| | | MTELPPTHPIRLGLALNFSVFYYEILNSPDRACRLAKAAFD | |
| | | DAIAELDTLSEESYKDSTLIMQLLRDNLTLWTSDMQGDGE | |
| | | EQNKEALQDVEDENQ | |
| M02 | Human/14-3-3 | MEKTELIQKAKLAEQAERYDDMATCMKAVTEQGAELSNE | 2 |
| | protein theta | ERNLLSVAYKNVVGGRRSAWRVISSIEQKTDTSDKKLQLIK | |
| | | DYREKVESELRSICTTVLELLDKYLIANATNPESKVFYLKM | |
| | | KGDYFRYLAEVACGDDRKQTIDNSQGAYQEAFDISKKEMQP | |
| | | THPIRLGLALNFSVFYYEILNNPELACTLAKTAFDEAIAE | |
| | | LDTLNEDSYKDSTLIMQLLRDNLTLWTSDSAGEECDAAEG | |
| | | AEN | |
| M03 | Human/14-3-3 | MGDREQLLQRARLAEQAERYDDMASAMKAVTELNEPLSN | 3 |
| | protein eta | EDRNLLSVAYKNVVGARRSSWRVISSIEQKTMADGNEKKL | |
| | | EKVKAYREKIEKELETVCNDVLSLLDKFLIKNCNDFQYES | |
| | | KVFYLKMKGDYYRYLAEVASGEKKNSVVEASEAAYKEAFE | |
| | | ISKEQMQPTHPIRLGLALNFSVFYYEIQNAPEQACLLAKQ | |
| | | AFDDAIAELDTLNEDSYKDSTLIMQLLRDNLTLWTSDQQD | |
| | | EEAGEGN | |
| M04 | Human/14-3-3 | MVDREQLVQKARLAEQAERYDDMAAAMKNVTELNEPLS | 4 |
| | protein gamma | NEERNLLSVAYKNVVGARRSSWRVISSIEQKTSADGNEKKI | |
| | | EMVRAYREKIEKELEAVCQDVLSLLDNYLIKNCSETQYES | |
| | | KVFYLKMKGDYYRYLAEVATGEKRATVVESSEKAYSEAH | |
| | | EISKEHMQPTHPIRLGLALNYSVFYYEIQNAPEQACHLAKT | |
| | | AFDDAIAELDTLNEDSYKDSTLIMQLLRDNLTLWTSDQQD | |
| | | DDGGEGNN | |
| M05 | Human/14-3-3 | MTMDKSELVQKAKLAEQAERYDDMAAAMKAVTEQGHEL | 5 |
| | protein | SNEERNLLSVAYKNVVGARRSSWRVISSIEQKTERNEKKQ | |
| | beta/alpha | QMGKEYREKIEAELQDICNDVLELLDKYLIPNATQPESKVF | |
| | | YLKMKGDYFRYLSEVASGDNKQTTVSNSQQAYQEAFEISK | |
| | | KEMQPTHPIRLGLALNFSVFYYEILNSPEKACSLAKTAFDE | |
| | | AIAELDTLNEESYKDSTLIMQLLRDNLTLWTSENQGDEGD | |
| | | AGEGEN | |
| M06 | Human/14-3-3 | MDKNELVQKAKLAEQAERYDDMAACMKSVTEQGAELSN | 6 |
| | protein | EERNLLSVAYKNVVGARRSSWRVVSSIEQKTEGAEKKQQ | |
| | zeta/delta | MAREYREKIETELRDICNDVLSLLEKFLIPNASQAESKVFYL | |
| | | KMKGDYYRYLAEVAAGDDKKGIVDQSQQAYQEAFEISKK | |
| | | EMQPTHPIRLGLALNFSVFYYEILNSPEKACSLAKTAFDEAI | |
| | | AELDTLSEESYKDSTLIMQLLRDNLTLWTSDTQGDEAEAG | |
| | | EGGEN | |
| M07 | Saccharomyces | MSQTREDSVYLAKLAEQAERYEEMVENMKAVASSGQELS | 7 |
| | cerevisiae/BM | VEERNLLSVAYKNVIGARRASWRIVSSIEQKEESKEKSEHQ | |
| | H2 | VELIRSYRSKIETELTKISDDILSVLDSHLIPSATTGESKVFYY | |
| | | KMKGDYHRYLAEFSSGDAREKATNSSLEAYKTASEIATTE | |
| | | LPPTHPIRLGLALNFSVFYYEIQNSPDKACHLAKQAFDDAIA | |
| | | ELDTLSEESYKDSTLIMQLLRDNLTLWTSDISESGQEDQQQ | |
| | | QQQQQQQQQQQQAPAEQTQGEPTK | |
| M08 | Human/DLG4 | MNPKRGFYIRALFDYDKTKDCGFLSQALSFRFGDVLHVID | 8 |
| | | ASDEEWWQARRVHSDSETDDIGFIPSKRRVERREWSRLKA | |
| | | KDWGSSSGSQGREDSVLSYETVTQMEVHYARPIIILGPTKD | |
| | | RANDDLLSEFPDKFGSCVPHTTRPKREYEIDGRDYHFVSSR | |
| | | EKMEKDIQAHKFIEAGQYNSHLYGTSVQSVREVAEQGKHC | |
| | | ILDVSANAVRRLQAAHLHPIAIFIRPRSLENVLEINKRITEEQ | |
| | | ARKAFDRATKLEQEFTECFSAIVEGDSFEEIYHKVKRVIEDL | |
| | | SGPYIWVPARERL | |
| M09 | Human/BRCA | MVNKRMSMVVSGLTPEEFMLVYKFARKHHITLTNLITEET | 9 |
| | 1 Tandem | THVVMKTDAEFVCERTLKYFLGIAGGKWVVSYFWVTQSI | |
| | domain | KERKMLNEHDFEVRGDVVNGRNHQGPKRARESQDRKIFR | |
| | | GLEICCYGPFTNMPTDQLEWMVQLCGASVVKELSSFTLGT | |
| | | GVHPIVVVQPDAWTEDNGFHAIGQMCEAPVVTREWVLDS | |
| | | VALYQCQELDTYLIPQIP | |
| M10 | Human/MCPH | MSGRGKKPTRTLVMTSMPSEKQNVVIQVVDKLKGFSIAPD | 10 |
| | 1 | VCETTTHVLSGKPLRTLNVLLGIARGCWVLSYDWVLWSLE | |
| | | LGHWISEEPFELSHHFPAAPLCRSECHLSAGPYRGTLFADQ | |
| | | PAMFVSPASSPPVAKLCELVHLCGGRVSQVPRQASIVIGPY | |
| | | SGKKKATVKYLSEKWVLDSITQHKVCAPENYLLSQ | |
| M11 | Human/TOPB | MVYNMVMSDVTISCTSLEKEKREEVHKYVQMMGGRVYR | 11 |
| | P1 | DLNVSVTHLIAGEVGSKKYLVAANLKKPILLPSWIKTLWE | |
| | | KSQEKKITRYTD | |
| M12 | Mycobacterium | MGSAGTSVTLQLDDGSGRTYQLREGSNIIGRGQDAQFRLP | 12 |
| | tuberculosis | DTGVSRRHLEIRWDGQVALLADLNSTNGTTVNNAPVQEW | |
| | /FhaA | QLADGDVIRLGHSEIIVRMH | |
| M13 | Saccharomyces | MENITQPTQQSTQATQRFLIEKFSQEQIGENIVCRVICTTGQI | 13 |
| | cerevisiae | PIRDLSADISQVLKEKRSIKKVWTFGRNPACDYHLGNISRLS | |
| | /FHA1 | NKHFQILLGEDGNLLLNDISTNGTWLNGQKVEKNSNQLLS | |
| | | QGDEITVGVGVESDILSLVIFINDKFKQCLEQNKVDRIRSNL | |
| | | KNTSKIASPGLTSSTASSMVANKTGIFKDFSIIDEVVGQGAF | |
| | | ATVKKAIERTTGKTFAVKIISKRKVIGNMDGVTRELEVLQK | |
| | | LNHPRIVRLKGFYEDTESYYMVMEFVSGG | |
| M14 | Saccharomyces | MEDQDGKIQGFKIPAHAPIRYTQPKSIEAETREQKLLHSNN | 14 |
| | cerevisiae | TENVKSSKKKGNGRFLTLKPLPDSIIQESLEIQQGVNPFFIGR | |
| | /RAD53 | SEDCNCKIEDNRLSRVHCFIFKKRHAVGKSMYESPAQGLD | |
| | | DIWYCHTGTNVSYLNNNRMIQGTKFLLQDGDEIKIIWDKN | |
| | | NKFVIGFKVEINDTTGLFNEGLGMLQEQRVVLKQTAEEKD | |
| | | LVKKLTQMMAAQRANQPSASSSSMSAKKPPVSDTNNNGN | |
| | | NSVLNDLVESPINANTGNILKRIHSVSLSQSQIDPSKKVKRA | |
| | | KLDQTSKGPENLQFS | |
| M15 | Human/Pin1 | MADEEKLPPGWEKRMSRSSGRVYYFNHITNASQWERPSG | 15 |
| | | NSSSGGKNGQGEPARVRCSHLLVKHSQSRRPSSWRQEKIT | |
| | | RTKEEALELINGYIQKIKSGEEDFESLASQFSDCSSAKARGD | |
| | | LGAFSRGQMQKPFEDASFALRTGEMSGPVFTDSGIHIILRTE | |
| M16 | Human/PLK1 | MSIAPSSLDPSNRKPLTVLNKGLENPLPERPREKEEPVVRET | 16 |
| | | GEVVDCHLSDMLQQLHSVNASKPSERGLVRQEEAEDPACI | |
| | | PIFWVSKWVDYSDKYGLGYQLCDNSVGVLFNDSTRLILYN | |
| | | DGDSLQYIERDGTESYLTVSSHPNSLMKKITLLKYFRNYMS | |
| | | EHLLKAGANITPREGDELARLPYLRTWFRTRSAIILHLSNGS | |
| | | VQINFFQDHTKLILCPLMAAVTYIDEKRDFRTYRLSLLEEY | |
| | | GCCKELASRLRYARTMVDKLLSSRSASNRLKAS | |
| M17 | Human/DIDO1 | MTLFLSRLSTIWKGFINMQSVAKFVTKAYPVSGCFDYLSED | 17 |
| | | LPDTIHIGGRIAPKTVWDYVGKLKSSVSKELCLIRFHPATEE | |
| | | EEVAYISLYSYFSSRGRFGVVANNNRHVKDLYLIPLSAQDP | |
| | | VPSKLLPFEGPGLESPRPNIILGLVICQKIKRPANS | |
| M18 | Human/MINT | MVDMVQLLKKYPIVWQGLLALKNDTAAVQLHFVSGNNV | 18 |
| | | LAHRSLPLSEGGPPLRIAQRMRLEATQLEGVARRMTVETD | |
| | | YCLLLALPCGRDQEDVVSQTESLKAAFITYLQAKQAAGIIN | |
| | | VPNPGSNQPAYVLQIFPPCEFSESHLSRLAPDLLASISNISPH | |
| | | LMIVIASV | |
| M19 | Human/RBM15 | MAPVASASPKLCLAWQGMLLLKNSNFPSNMHLLQGDLQV | 19 |
| | | ASSLLVEGSTGGKVAQLKITQRLRLDQPKLDEVTRRIKVAG | |
| | | PNGYAILLAVPGSSDSRSSSSSAASDTATSTQRPLRNLVSYL | |
| | | KQKQAAGVISLPVGGNKDKENTGVLHAFPPCEFSQQFLDS | |
| | | PAKALAKSEEDYLVMIIVRGFGFQI | |
| M20 | Human/RB15B | MQLGWNGLLVLKNSCFPTSMHILEGDQGVISSLLKDHTSG | 20 |
| | | SKLTQLKIAQRLRLDQPKLDEVTRRIKQGSPNGYAVLLATQ | |
| | | ATPSGLGTEGMPTVEPGLQRRLLRNLVSYLKQKQAAGVIS | |
| | | LPVGGSKGRDGTGMLYAFPPCDFSQQYLQSALRTLGKLEE | |
| | | EHMVIVIVRDTA | |
| M21 | Human/PHF3 | MTFLARLNFIWKGFINMPSVAKFVTKAYPVSGSPEYLTEDL | 21 |
| | | PDSIQVGGRISPQTVWDYVEKIKASGTKEICVVRFTPVTEED | |
| | | QISYTLLFAYFSSRKRYGVAANNMKQVKDMYLIPLGATDK | |
| | | IPHPLVPFDGPGLELHRPNLLLGLIIRQKL | |
| M22 | Schizosaccharomyces | MKQKVLKGCRLLFSGVIPLGVDVLSSDIAKWAMSFGAEVV | 22 |
| | pombe | LDFSVPPTHLIAAKIRTEKVKKAVSMGNIKVVKLNWLTESL | |
| | /FCP1 | SQWKRLPESDYLL | |
| M23 | Human/Scp1 | MAFEELRVQAENARLDMHFKLKEDHEKIQHLQEEYKKEV | 23 |
| | | NDKENQVSLLLIQRTEKENKMKDLTFLLEESRDKVNQLED | |
| | | KTKLQDENVKELNKKKDHLTSELEDTKMSLQRSMNTQKA | |
| | | LEEDLQIATKTIYQLTEEKEAQMEEFNKAKTDHSFMVTELK | |
| | | ATTCTLEELLRTEQQRLVKNED | |
| M26 | Tandem SH2 | MGIDPFTAKRTHRVINHPYYFPFNGRQAEDYLRSKERGEFV | 24 |
| | domain | IRQSSRGDDHLVITWKLDKDLFQHIDIQELEKENPLALGKV | |
| | S. cerevisiae | LIVDNQKYNDLDQIIVEYLQNKVRLLNEMTSSEKFKSGTKK | |
| | | DVVKFIEDYSRVNPNKSVYYFSLNHDNPGWFYLMFKINAN | |
| | | SKLYTWNVKLTNTGYFLVNYNYPSVIQLCNGFKTLLKSNS | |
| | | SKNRMNNYR | |
| M27 | SCOP domain | MSGLVPRGSHMASGPPDSDHIWRGMIAKGGTPVCCARCVP | 25 |
| | FPA | MGKGIETKLPEVVNCSARTDLNMLAKHYAVAIGCEIVFFV | |
| | arabidopsis | PDREEDFASYTEFLRYLSSKDRAGVAKLDDGTTLFLVPPSD | |
| | | FLTDVLQVTRQERLYGVVLKLPPPA | |
| M28 | SCOP domain | MGAMGSTFLARLNFIWKGFINMPSVAKFVTKAYPVSGSPE | 26 |
| | PHF3 human | YLTEDLPDSIQVGGRISPQTVWDYVEKIKASGTKEICVVRF | |
| | | TPVTEEDQISYTLLFAYFSSRKRYGVAANNMKQVKDMYLIP | |
| | | LGATDKIPHPLVPFDGPGLELHRPNLLLGLIIRQKLKRQHS | |
| M29 | PPM | MARKKYMEISLLTDIGQRRSNNQDFINQFENKAGVPLIILA | 27 |
| | streptococcus | DGMGGHRAGNIASEMTVTDLGSDWAETDFSELSEIRDWML | |
| | agalacticae | VSIETENRKIYELGQSDDYKGMGTTIEAVAIVGDNIIFAHV | |
| | | GDSRIGIVRQGEYHLLTSDHSLVNELVKAGQLTEEEAASHP | |
| | | QKNIITQSIGQANPVEPDLGVHLLEEGDYLVVNSDGLTNML | |
| | | SNADIATVLTQEKTLDDKNQDLITLANHRGGLDNITVALV | |
| | | YVESEAV | |
| M30 | PP5 | MKNAFKGAKIKNMSQEFISKMVNDLFLKGKYLPKKYVAAI | 28 |
| | saccharomyces | ISHADTLFRQEPSMVELENNSTPDVKISVCGDTHGQFYDV | |
| | cerevisae | LNLFRKFGKVGPKHTYLFNGDFVDRGSWSCEVALLFYCLK | |
| | | ILHPNNFFLNRGNHESDNMNKIYGFEDECKYKYSQRIFNM | |
| | | FAQSFESLPLATLINNDYLVMHGGLPSDPSATLSDFKNID | |
| | | RFAQPPRDGAFMELLWADPQEANGMGPSQRGLGHAFGPDI | |
| | | TDRFLRNNKLRKIFRSHELRMGGVQFEQKGKLMTVFSAPN | |
| | | YCDSQGNLGGVIHVVPGHGILQAGRNDDQNLIIETFEAVE | |
| | | HPDIKPMAYSNGGFGL | |
| M31 | PP2A human | MDEKVFTKELDQWIEQLNECKQLSESQVKSLCEKAKEILT | 29 |
| | | KESNVQEVRCPVTVCGDVHGQFHDLMELFRIGGKSPDTNY | |
| | | LFMGDYVDRGYYSVETVTLLVALKVRYRERITILRGNHES | |
| | | RQITQVYGFYDECLRKYGNANVWKYFTDLFDYLPLTALVD | |
| | | GQIFCLHGGLSPSIDTLDHIRALDRLQEVPHEGPMCDLLW | |
| | | SDPDDRGGWGISPRGAGYTFGQDISETFNHANGLTLVSRA | |
| | | HQLVMEGYNWCHDRNVVTIFSAPNYCYRCGNQAAIMELD | |
| | | DTLKYSFLQFDPAPRRGEPHVTRRTPDYFL | |
| M32 | PP1 rabbit | MSDSEKLNLDSIIGRLLEVQGSRPGKNVQLTENEIRGLCLKS | 30 |
| | | REIFLSQPILLELEAPLKICGDIHGQYYDLLRLFEYGGFPPE | |
| | | SNYLFLGDYVDRGKQSLETICLLLAYKIKYPENFFLLRGNHE | |
| | | CASINRIYGFYDECKRRYNIKLWKTFTDCFNCLPIAAIVDEK | |
| | | IFCCHGGLSPDLQSMEQIRRIMRPTDVPDQGLLCDLLWSDP | |
| | | DKDVQGWGENDRGVSFTFGAEVVAKFLHKHDLDLICRAH | |
| | | QVVEDGYEFFAKRQLVTLFSAPNYCGEFDNAGAMMSVDE | |
| | | TLMCSFQILKPADKNKGKYGQFSGLNPGGRPITPPRNSAKA | |
| | | KK | |
| M33 | PP1 rabbit | MSDSEKLNLDSIIGRLLEVQGSRPGKNVQLTENEIRGLCLKS | 31 |
| | D95N | REIFLSQPILLELEAPLKICGDIHGQYYDLLRLFEYGGFPPE | |
| | | SNYLFLGDYVNRGKQSLETICLLLAYKIKYPENFFLLRGNHE | |
| | | CASINRIYGFYDECKRRYNIKLWKTFTDCFNCLPIAAIVDEK | |
| | | IFCCHGGLSPDLQSMEQIRRIMRPTDVPDQGLLCDLLWSDP | |
| | | DKDVQGWGENDRGVSFTFGAEVVAKFLHKHDLDLICRAH | |
| | | QVVEDGYEFFAKRQLVTLFSAPNYCGEFDNAGAMMSVDE | |
| | | TLMCSFQILKPADKNKGKYGQFSGLNPGGRPITPPRNSAKA | |
| | | KK | |
| M34 | PPZ1 candida | MGHMIDIDSLIDKLLNAGFSGKRTKNVCLKNTEIELICASA | 32 |
| | albicans | REIFLSQPSLLELAPPVKVVGDVHGQYHDLIRIFSKCGFPPK | |
| | | TNYLFLGDYVNRGKQSLETILLLLCYKIKYPENFFLLRGNH | |
| | | ECANVTRVYGFYDECKRRCNIKTWKLFIDTENTLPIAAIVA | |
| | | GKIFCVHGGLSPVLNSMDEIRNIARPTDVPDFGLLNDLLWS | |
| | | DPADTINEWEDNERGVSYVFSKVAINKFLSKFNFDLVCRA | |
| | | HMVVEDGYEFFNDRTLVTVFSAPNYCGEFDNWGAVMGVS | |
| | | EDLLCSFELLDPLDSAALKQVMKKEKQ | |
| M47 | Wild type Fyn | MVAPVDSIQAEEWYFGKLGRKDAERQLLSFGNPRGTFLIR | 33 |
| | SH2 domain | ESETTKGAYSLSIRDWDDMKGDHVKHYKIRKLDNGGYYIT | |
| | | TRAQFETLQQLVQHYSERAAGLSSRLVVPSHKGGGSHHHH | |
| | | qHHHHHHGGGSGGGSGGGSGLNDFFEAQKIEWHEGGGSG | |
| | | GGSGGGSGLNDFFEAQKIEWHE | |
| M48 | Wild Type Src | MDSIQAEEWYFGKITRRESERLLLNAENPRGTFLVRESET | 34 |
| | SH2 domain | TKGAYCLSVSDFDNAKGLNVKHYKIRKLDSGGFYITSRTQ | |
| | | FNSLQQLVAYYSKHADGLSHRLTTVSPTSGGSHHHHHHHH | |
| | | HHGGGSGGGSGGGSGLNDFFEAQKIEWHEGGGSGGGSGG | |
| | | GSGLNDFFEAQKIEWH | |
| S33 | SH domanin | MGAMDSIQAEEWYFGKLGRKDAERQLLSFGNPRGTFLIRE | 35 |
| | Fyn Triple | SETVKGAYALSIRDWDDMKGDHVKHYLIRKLDNGGYYIT | |
| | mutant | TRAQFETLQQLVQHYSERAAGLSSRLVVPSHK | |

### Amino Acid Recognition Molecules

An amino acid recognition molecule (e.g., terminal amino acid recognition molecule) is a molecule that specifically binds to a certain amino acid (e.g., binds to the certain amino acid with a higher affinity than any other amino acid). Amino acid recognition molecules include, for example, proteins and nucleic acids, which may be synthetic or recombinant. In some embodiments, an amino acid recognition molecule may be an antibody or an antigen-binding portion of an antibody, an SH2 domain-containing protein or fragment thereof, an FHA domain-containing protein or fragment thereof, or an enzymatic biomolecule, such as a peptidase, an aminotransferase, a ribozyme, an aptazyme, or a tRNA synthetase, including aminoacyl-tRNA synthetases and related molecules described in U.S. patent application Ser. No. 15/255,433, filed Sep. 2, 2016, titled “MOLECULES AND METHODS FOR ITERATIVE POLYPEPTIDE ANALYSIS AND PROCESSING.” In some embodiments, an amino acid recognition molecule is an antibody (e.g., a single-chain antibody variable fragment (scFv) or VHH (Nanobody). In some embodiments, an amino acid recognition molecule is an aptamer.

In some embodiments, an amino acid recognition molecule is a degradation pathway protein. Examples of degradation pathway proteins suitable for use as recognition molecules include, without limitation, N-end rule pathway proteins, such as Arg/N-end rule pathway proteins, Ac/N-end rule pathway proteins, and Pro/N-end rule pathway proteins. In some embodiments, an amino acid recognition molecule is an N-end rule pathway protein selected from a Gid protein (e.g., Gid4 or Gid10 protein), a UBR box protein (e.g., UBR1, UBR2) or UBR box domain-containing protein fragment thereof, a p62 protein or ZZ domain-containing fragment thereof, a ClpS protein (e.g., ClpS1, ClpS2), Baculoviral inhibitor of apoptosis (IAP) repeat-containing (BIR) protein (e.g., BIR3), an Ntaq1 protein, and a Zer/Zyg protein.

Examples of amino acid recognition molecules and uses thereof, including methods of polypeptide sequencing and other sequencing reagents (e.g., cleaving reagents) suitable for use in accordance with the disclosure have been described more fully, for example, in PCT International Publication Nos. WO2020102741A1, WO2021236983A2, WO2023122769A2, WO2024031031A2, and WO2024086832A1, each of which is incorporated by reference in its entirety.

### EXAMPLES

### Example 1

This Example describes an exemplary method of the disclosure for use in identifying the presence of a phosphorylated tyrosine on a model polypeptide.

A control polypeptide (LAQYLAYPDDDK) and a model polypeptide comprising a phospho-tyrosine (LAQ-pY-LAYPDDDK) were tested in this Example. Each polypeptide was first immobilized onto independent surfaces of a chip. A post-translational modification-specific (PTM-specific) affinity reagent that binds to phospho-tyrosine (PS33 (SEQ ID NO: 35)) and comprised a fluorescent label was then added to the chip and allowed to contact the polypeptides. A fluorescence signature representative of the binding interaction(s) between each polypeptide and the PTM-specific affinity reagent was collected using a detector for 30 minutes. The chip was then washed to remove the PTM-specific affinity reagent. A protein sequencing analysis of the polypeptides was then performed. A mixture of labeled terminal amino acid recognition molecules was added to the chip and allowed to incubate for 15 minutes. After 15 minutes, a mixture of cleaving reagents (a mixture of aminopeptidases) was added to the chip. During these steps, a series of signal pulses indicative of association of the one or more terminal amino acid recognition molecules with successive amino acids exposed at a terminus of each polypeptide were collected while each polypeptide was being degraded.

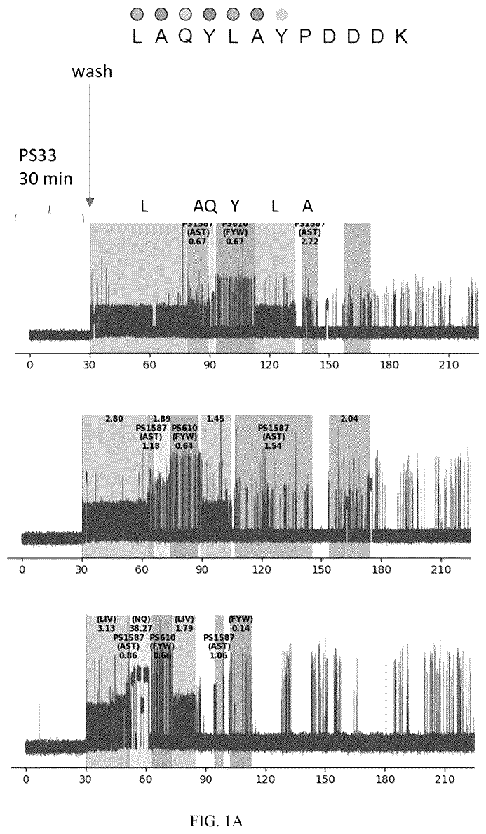

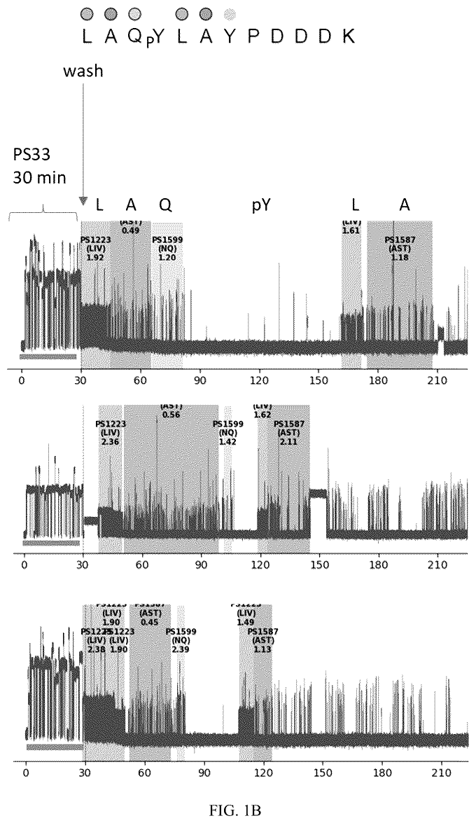

As shown in FIG. 1A, there was no fluorescence signature representative of binding interaction(s) between the polypeptide that did not include a PTM (LAQYLAYPDDDK) and the PTM-specific affinity reagent. On the contrary, there was a strong fluorescence signature representative of binding interaction(s) between the polypeptide that did include a PTM (LAQ-pY-LAYPDDDK) and the PTM-specific affinity reagent (FIG. 1B). In addition to identifying that the model included a phospho-tyrosine, the inventors were further able to determine that it was the more N-terminal tyrosine (i.e., the tyrosine at position 4 of the polypeptide) that was phosphorylated and not the second tyrosine (i.e., the tyrosine at position 7) based on the sequencing analysis. Note that, in this context, the sequencing analysis alone would not have identified that the amino acid at position 4 was a phospho-tyrosine. The terminal amino acid recognition molecule for tyrosine did not identify the amino acid at position 4 as being a tyrosine.

### Example 2

This Example demonstrates that methods of the disclosure can be used to identify a single post-translational modification within the context of a full-length natively folded protein. Such methods can be useful for determination of proteoforms in a sample (e.g., determination of proteoforms at a single-molecule level).

A full-length polypeptide (green fluorescent protein (GFP) was immobilized on a surface of a chip. A PTM-specific affinity reagent (a fluorescently labeled VHH nanobody that binds to a PTM) was added to the chip and allowed to interact with the full-length polypeptide under native conditions. The PTM-specific affinity reagent was added to a chip containing no immobilized polypeptide as a control experiment. A fluorescence signature representative of the binding interaction(s) between the polypeptide and the PTM-specific affinity reagent was collected using a detector for 30 minutes.

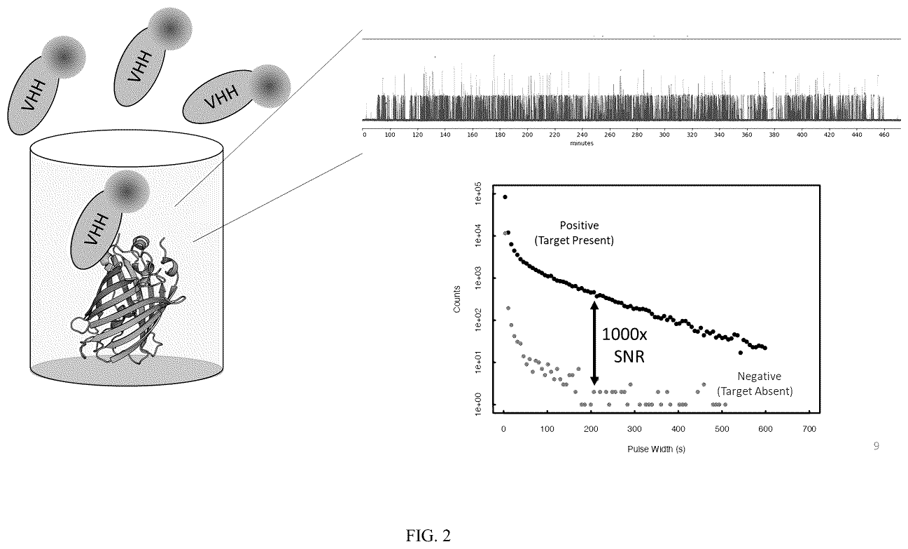

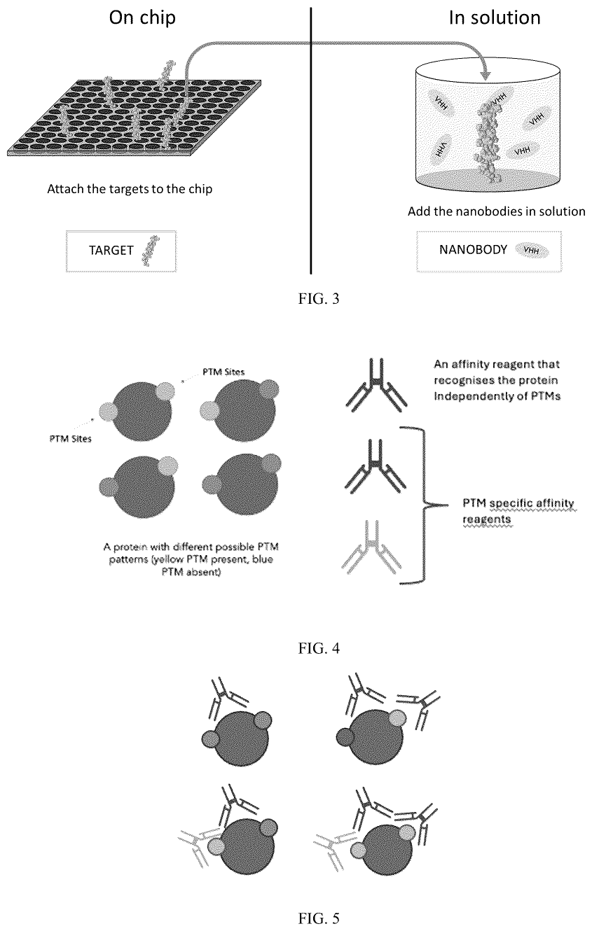

As shown in FIG. 2, there was a strong fluorescence signal detected in the experimental condition including the full-length polypeptide and the labeled PTM-specific affinity reagent. Conversely, there was minimal signal detected in the control containing only the labeled PTM-specific affinity reagent. The difference in signal detected provided a signal-to-noise ratio of approximately 1000, indicating that the method was very robust in its ability to determine that the full-length polypeptide comprised a PTM.

### Example 3

This Example characterizes an exemplary phosphorylated tyrosine recognizer for use in methods of the disclosure.

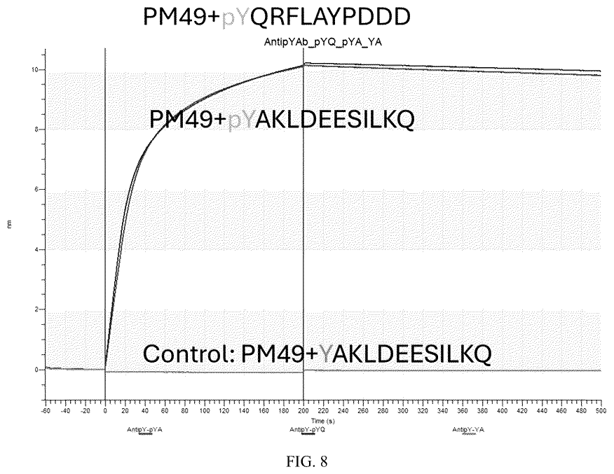

A control peptide (YAKLDEESILKQ) and two model peptides (pYQRFLAYPDDD and pYAKLDEESILKQ) were first tested in this Example to evaluate phospho-tyrosine detection in different peptide motifs using a post-translational modification-specific (PTM-specific) affinity reagent that binds to phospho-tyrosine (PM49 (ABCAM® Biotin Anti-Phosphotyrosine antibody [EPR16871])). Using the Octet binding platform, Octet sensors were coated with the peptide of interest and dipped in buffer containing the PTM-specific affinity reagent. The results showed a positive shift to 10.2 with both model peptides, indicating strong affinity of the PTM-specific affinity reagent to phospho-tyrosine in the peptides. In contrast, no shift was detected with the control peptide, indicating no affinity of the PTM-specific affinity reagent to unphosphorylated tyrosine in the peptides (FIG. 8).

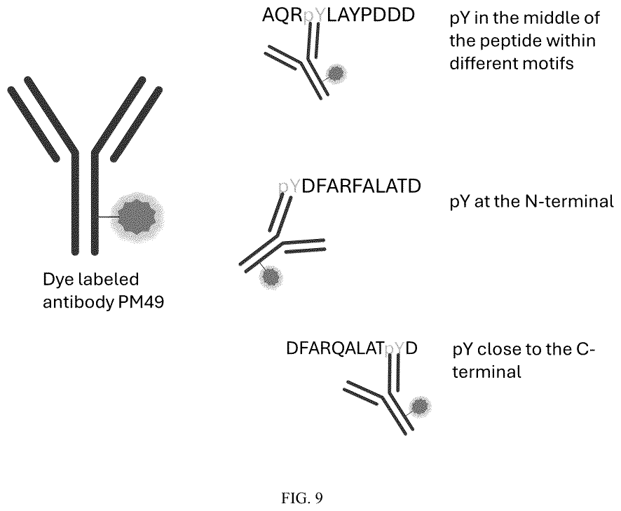

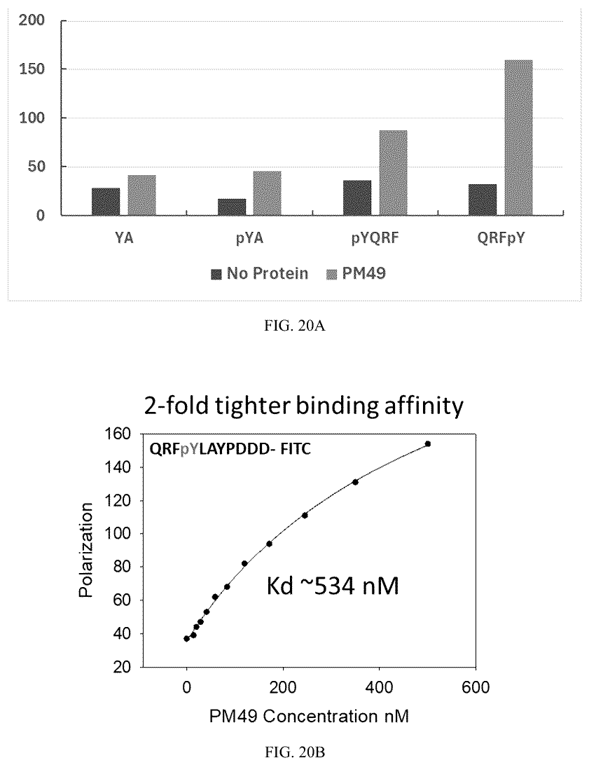

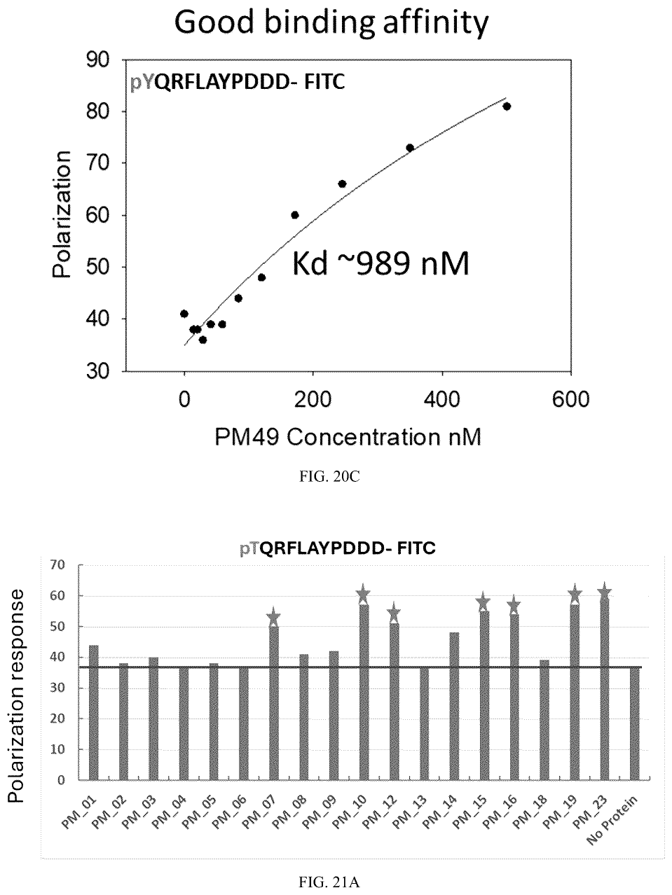

The phospho-tyrosine-specific affinity reagent, PM49, was evaluated for its ability to bind phospho-tyrosine residues residing in a range of motifs and at different positions in peptides (FIG. 9). First, bulk polarization binding affinity data was obtained using fixed concentrations of PM49, and various phospho-tyrosine peptides labeled with FITC. A control peptide without phospho-tyrosine modifications was also assayed. Binding affinity of PM49 to phospho-tyrosine was found to vary with peptide sequence and phospho-tyrosine location (FIG. 20A). For example, PM49 showed a 2-fold tighter binding affinity to an internal phospho-tyrosine (FIG. 20B) compared to an N-terminal phospho-tyrosine (FIG. 20C). This data suggests that phospho-tyrosine binding by PTM-specific affinity reagents could have distinct binding kinetics (e.g., pulse duration and interpulse duration), and could thus be distinguished, based on their location in the peptide and the peptide sequence itself.

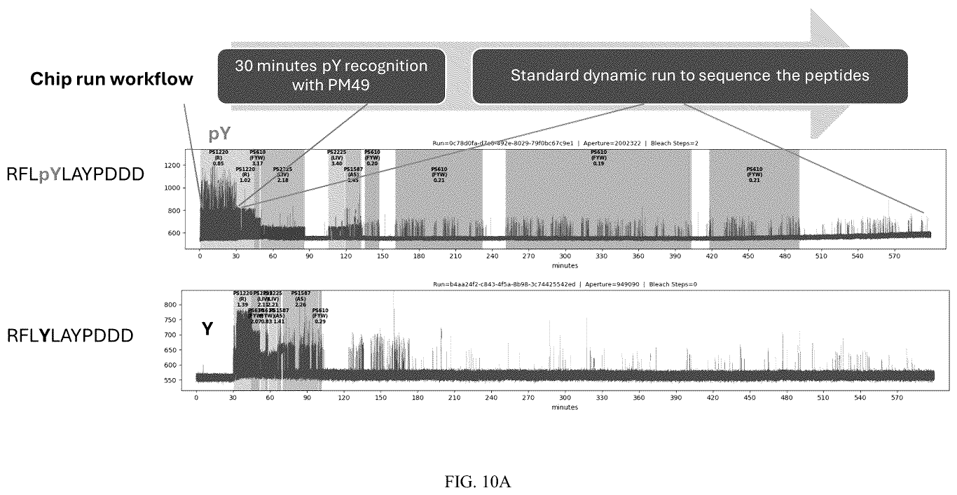

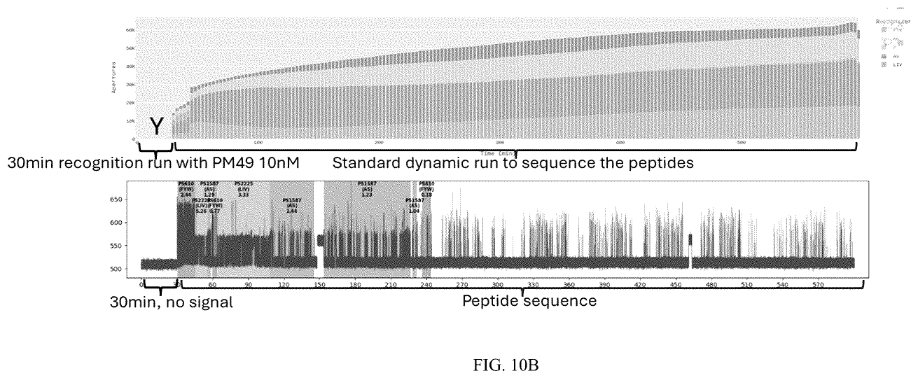

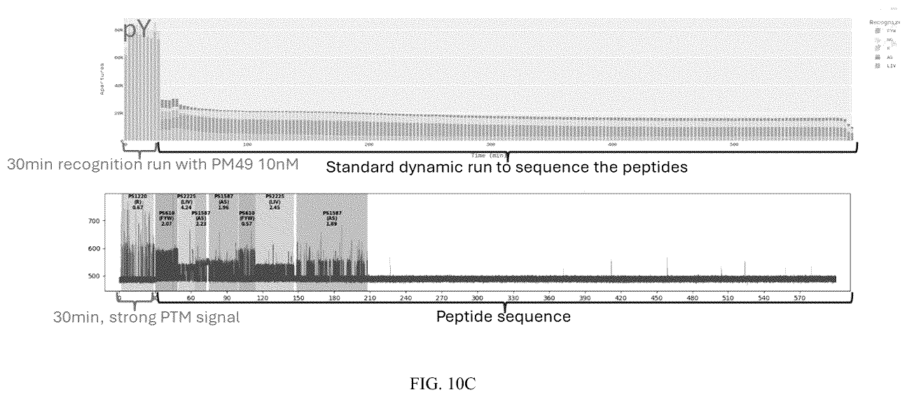

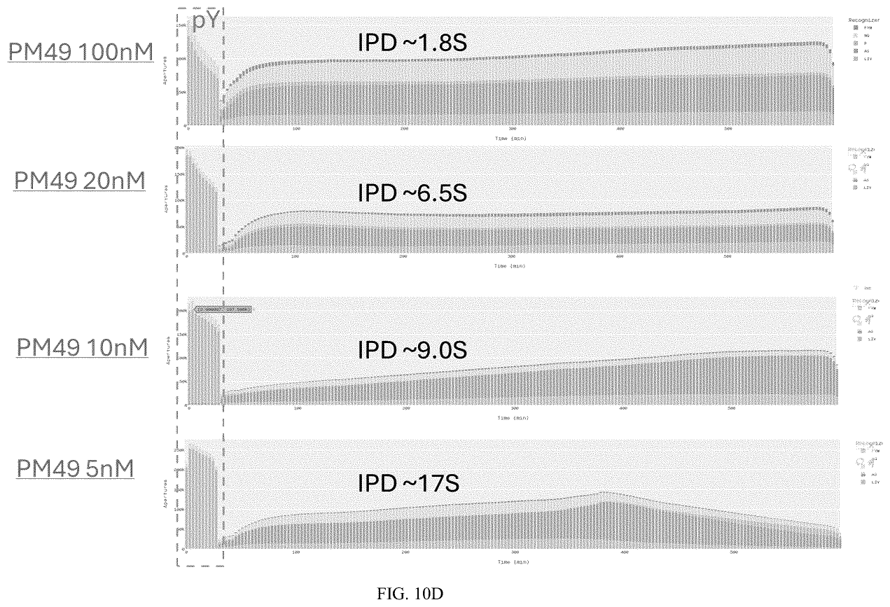

Next, two-stage on-chip recognition runs were performed in which the PTM-specific affinity reagent was incubated with a peptide for 30 minutes, followed by peptide sequencing. Using a model peptide (RFL-pY-LAYPDDD), fast pulsing derived from the interaction between the phospho-tyrosine and the PTM-specific affinity reagent was detected in the first 30 minutes. In contrast, no pulsing was detected in the first 30 minutes when using a control peptide that did not comprise a phospho-tyrosine (RFLYLAYPDDD). Peptide sequencing following PTM detection (FIGS. 10A-10D).

The example results shown in FIGS. 10A-10D demonstrated that two-stage recognition runs were successfully carried out in parallel at the single molecule level. As depicted by the workflow in FIG. 10A, the two-stage recognition runs included a first stage in which the PTM-specific affinity reagent was added to a chip to determine the presence or absence of phospho-tyrosine in single peptide molecules immobilized in individual apertures on the chip. This was followed by a second stage in which reagents for peptide sequencing (amino acid recognition molecules, cleaving reagents) were added to the chip to determine sequence information for the single peptide molecules. The plots shown at the top of FIGS. 10B and 10C are stacked plots showing the number of apertures (y-axis) with recognition taking place on the chip over the course of two-stage recognition runs (x-axis) performed in parallel with peptides having unphosphorylated tyrosine (FIG. 10B) or phosphorylated tyrosine (FIG. 10C). Each plot shown at the bottom of FIGS. 10B and 10C is an example binding trace from a single aperture on the chip. As shown in the bottom plot of FIG. 10C, both stages of the recognition run with phospho-tyrosine peptide displayed signal pulsing indicative of on/off binding events between the peptide and the reagents used for PTM detection and peptide sequencing. Additionally, a range of PTM-specific affinity reagent concentrations were found to yield sufficient pulses for phospho-tyrosine recognition and demonstrated that the interpulse duration was concentration dependent, with lower concentrations of the PTM-specific affinity reagent showing slower on-rates (FIG. 10D).

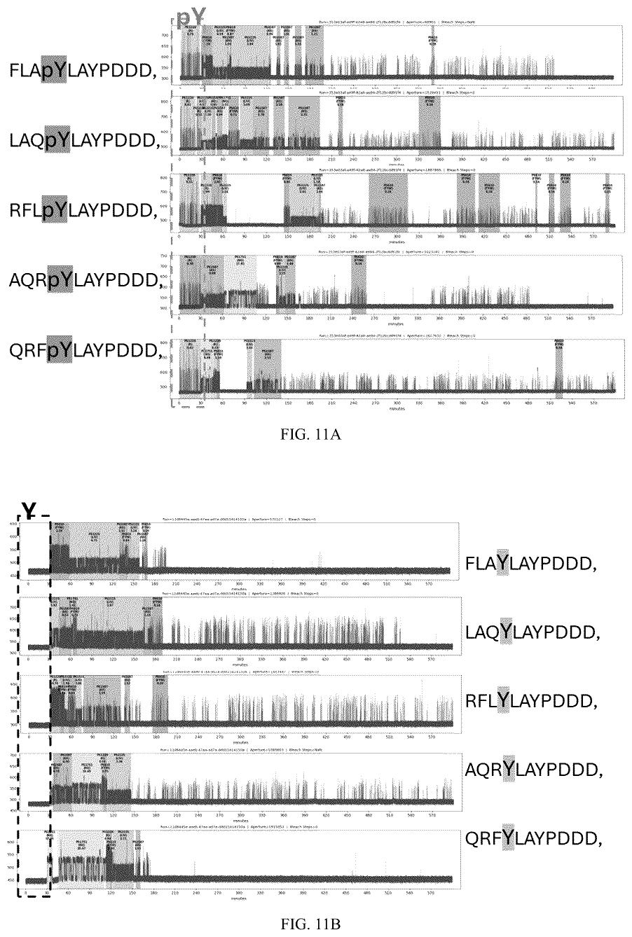

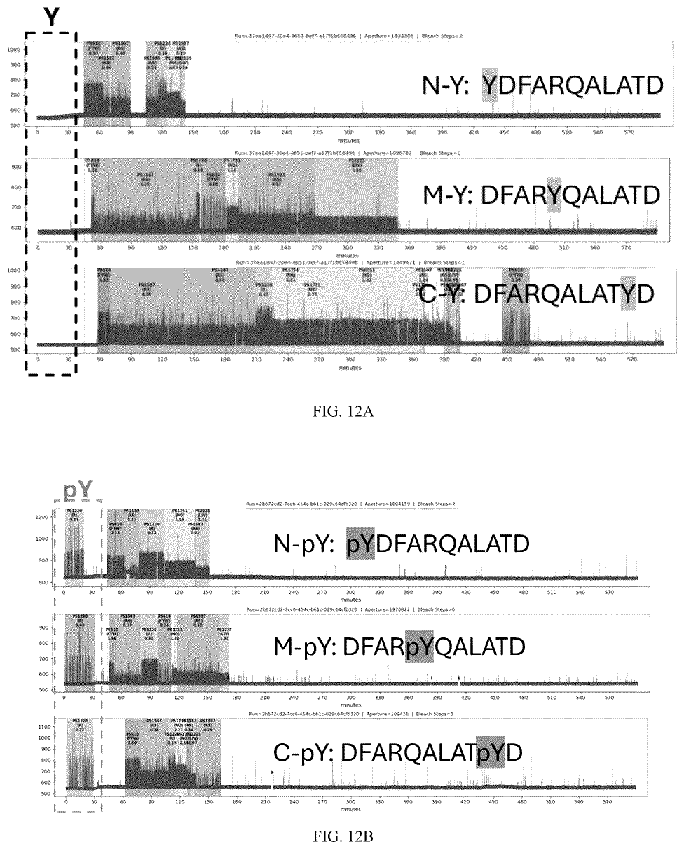

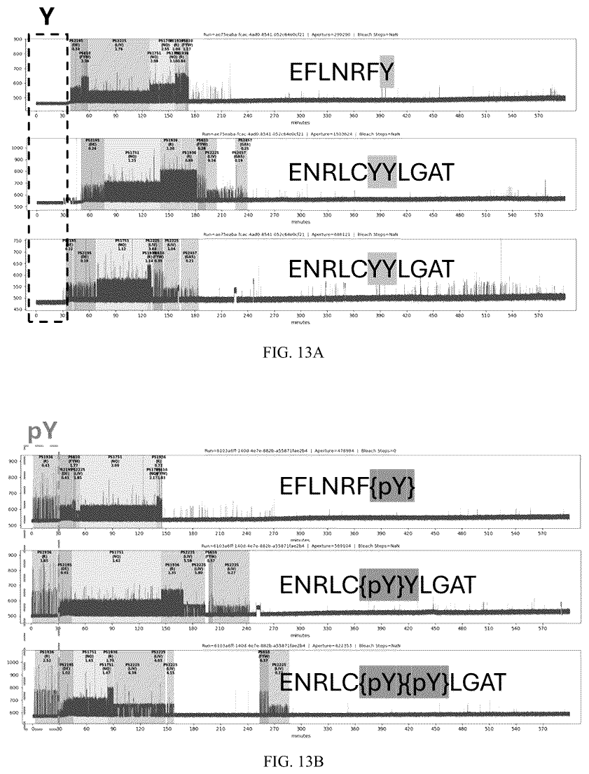

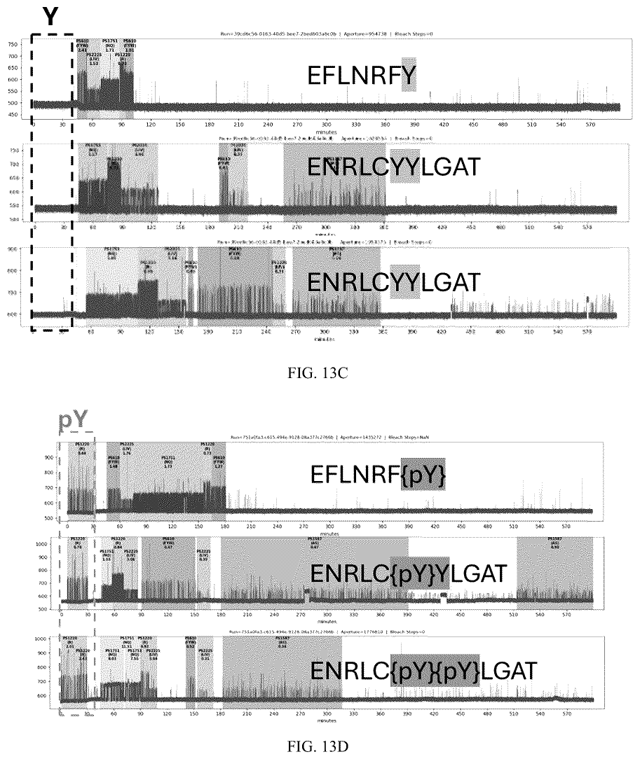

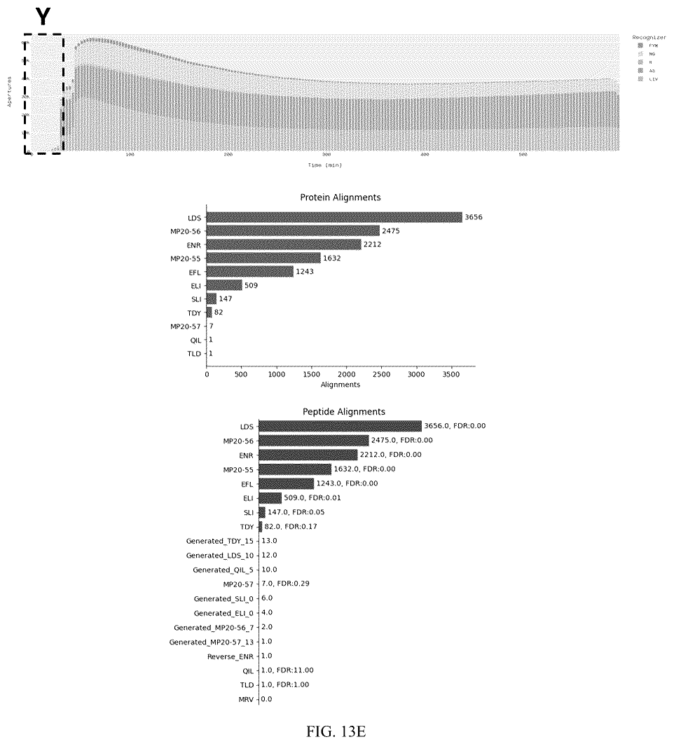

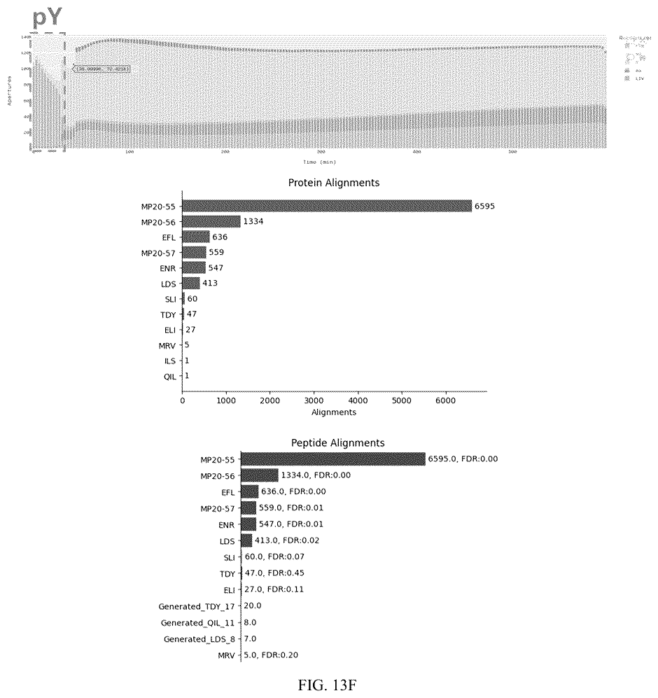

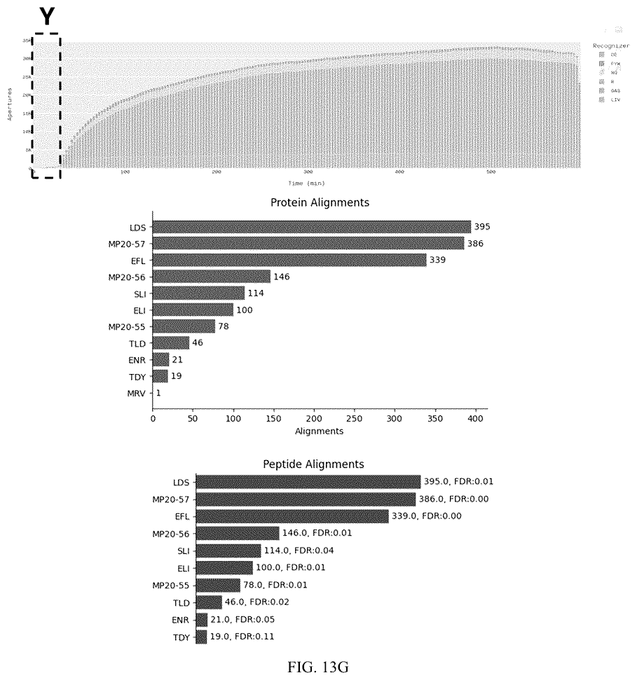

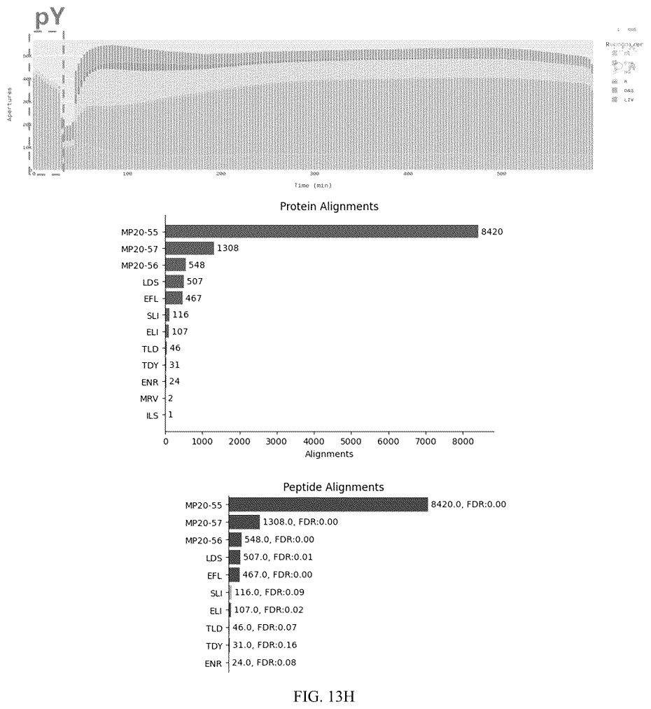

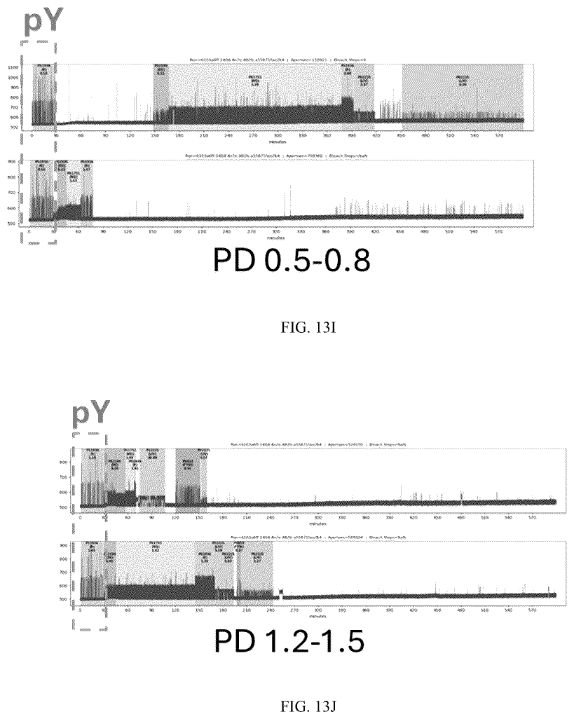

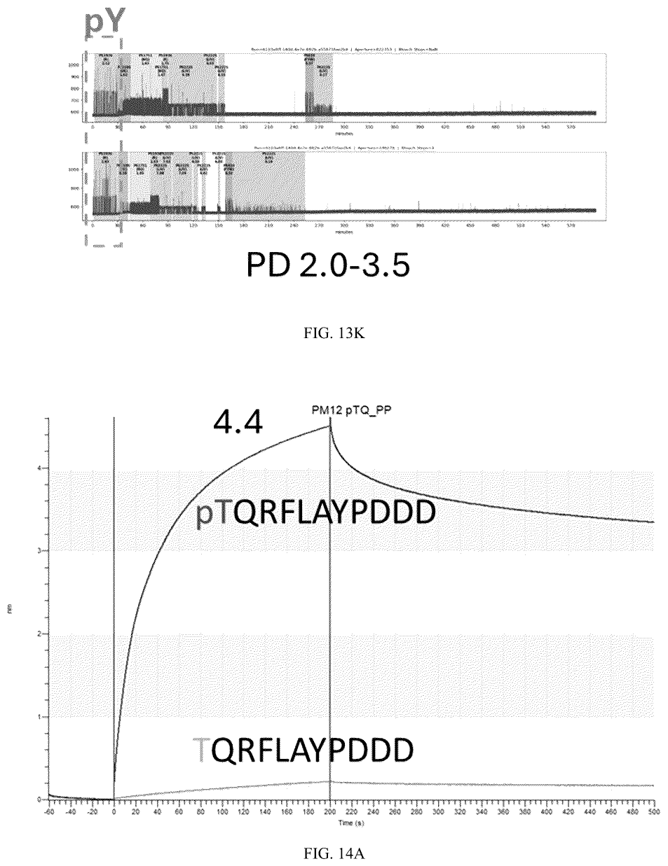

Using the two-stage recognition run format described above, PTM recognition of phospho-tyrosine in various peptide motifs was evaluated. Phospho-tyrosine detection by the PTM-specific affinity reagent in different peptide motifs was evaluated with unphosphorylated tyrosine (FLAYLAYPDDD, LAQYLAYPDDD, RFLYLAYPDDD, AQRYLAYPDDD, and QRFYLAYPDDD) and compared to their phosphorylated tyrosine counterparts (FLA-pY-LAYPDDD, LAQ-PY-LAYPDDD, RFL-pY-LAYPDDD, AQR-pY-LAYPDDD, and QRF-pY-LAYPDDD). The PTM-specific affinity reagent was able to detect phospho-tyrosine in all five peptide motifs tested, with pulsing detected in the first 30 minutes of each run (FIG. 11A). In contrast, no signal was detected for any of the unphosphorylated peptide motifs (FIG. 11B). The PTM-specific affinity reagent was also evaluated for its ability to detect phospho-tyrosine residues at different positions in peptides. Pulsing in the first 30 minutes demonstrated that the PTM-specific affinity reagent recognized phospho-tyrosine residues at the N-terminus, the C-terminus, and in the middle of peptides, while no signal was observed for unphosphorylated tyrosine residues at corresponding positions in peptides (FIGS. 12A-12B). Similar results were obtained by performing the same two stage chip run on phospho-tyrosine peptides spiked in the CDNF libraries (FIGS. 13A-13D). Core analysis plots and protein alignments of the two stage chip run on phospho-tyrosine peptides spiked in the CDNF libraries demonstrated that the PTM-specific affinity reagent binds to phospho-tyrosine readily binds to spiked in CDNF peptides (MP20-55: EFLNRF{pY}K; MP20-56: ENRLC{pY}YLGATK, MP20-57 ENRLC{pY}{pY}LGATK) and not to any peptides in the CDNF library (FIGS. 13E-13H). Additionally, traces of the spiked in CDNF peptide revealed that different motifs (e.g., pY, pYY, and pYpY) could be distinguished by their pulse duration (FIGS. 13I-13K).

This Example demonstrates the use of a PTM-specific affinity reagent to identify phospho-tyrosine residues in different peptide motifs at the N-terminus, C-terminus, and middle of the peptide. The N-terminus pY show faster pulsing than C-terminus, and middle of a peptide during the pre-sequencing stage of the recognition run. This Example further demonstrates the use of a PTM-specific affinity reagent in a two stage chip run for sequential proteoform identification and peptide sequencing.

### Example 4

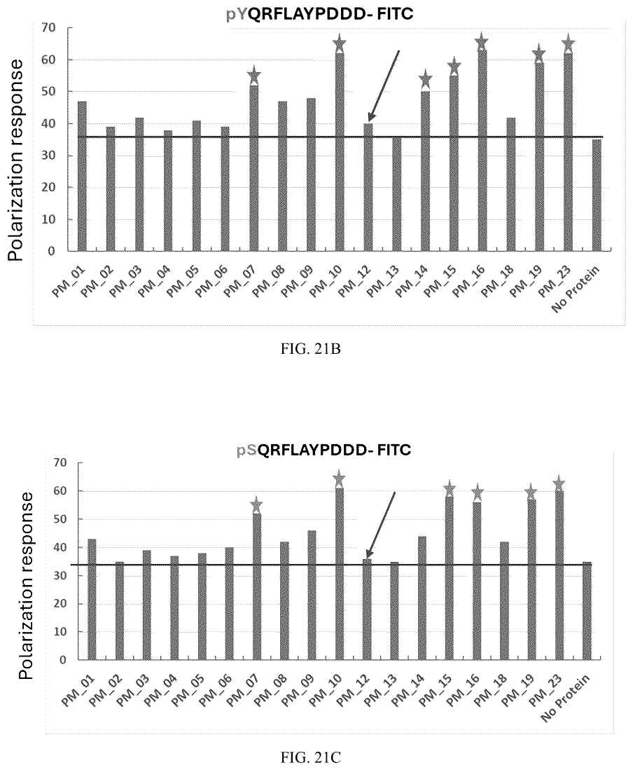

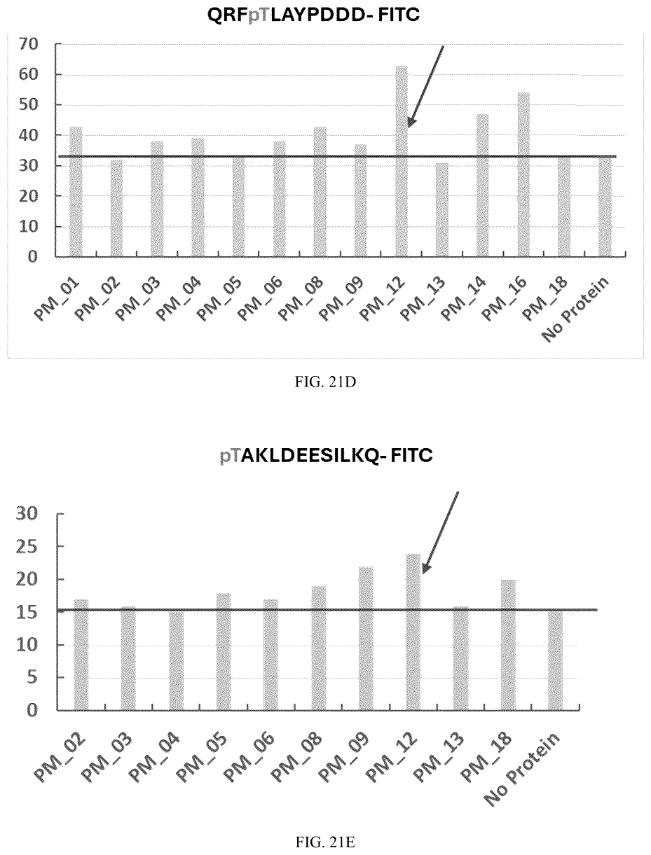

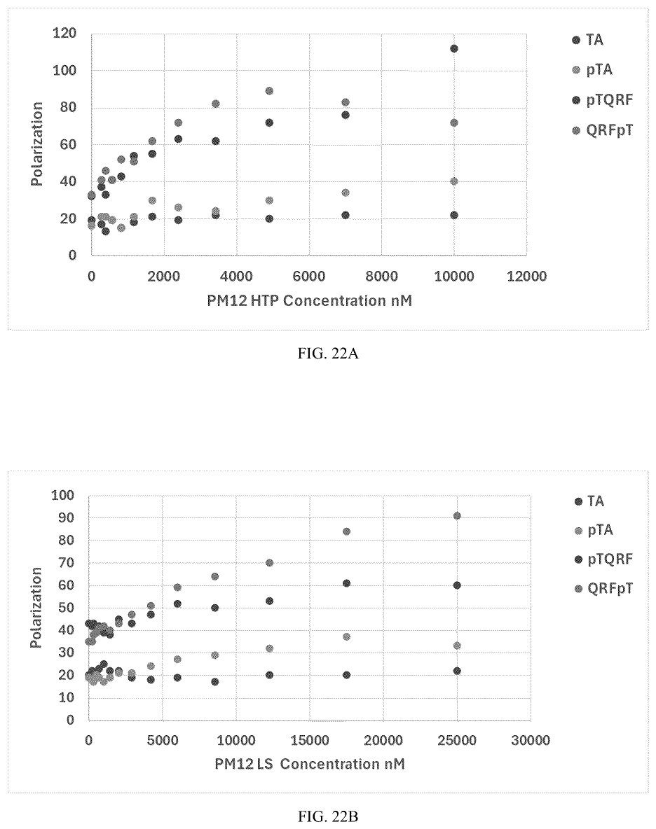

This Example characterizes an exemplary phosphorylated threonine recognizer for use in methods of the disclosure. Octet binding assays at a fixed concentration of PTM-specific affinity reagent candidates were evaluated for binding with phospho-threonine, phospho-serine containing peptides (pSAKLDEESILKQ-Biotin; pTAKLDEESILKQ-Biotin; pTQRFLAYPDDD-Biotin), and control peptides without modifications (SAKLDEESILKQ-Biotin; TAKLDEESILKQ-Biotin). The Octet binding assay responses are shown in Table 2 and FIGS. 21A-21E. PM12 (SEQ ID NO: 12) was identified as having high specificity for phospho-threonine, and low binding for phospho-tyrosine and phospho-serine, and was able to bind to phospho-threonine in different peptide sequences and at different locations in the peptide, with a slight preference for internal phospho-threonines compared to N-terminal phospho-threonines (FIGS. 22A-22B). This data suggests that phospho-threonine binding by PTM-specific affinity reagents could have distinct binding kinetics (e.g., pulse duration and interpulse duration), and could thus be distinguished, based on their location in the peptide and the peptide sequence itself.

TABLE 2
Octet binding assay responses for PTM-
specific affinity reagent candidates.

| Description | Source | pSA | SA | pTQR | pTA | TA |
|---|---|---|---|---|---|---|
| PM01 | Human/14-3-3 | 0.2 | 0.2 | — | — | — |
| | protein epsilon | | | | | |
| PM02 | Human/14-3-3 | 0.3 | 0.3 | 0.5 | 0.3 | 0.3 |
| | protein theta | | | | | |
| PM03 | Human/14-3-3 | 0.2 | 0.3 | 0.4 | 0.2 | 0.2 |
| | protein eta | | | | | |
| PM04 | Human/14-3-3 | 0.1 | 0.1 | — | — | — |
| | protein gamma | | | | | |
| PM05 | Human/14-3-3 | 0.2 | 0.2 | 0.2 | 0.2 | 0.2 |
| | protein beta/alpha | | | | | |
| PM06 | Human/14-3-3 | 0.1 | 0.1 | — | — | — |
| | protein zeta/delta | | | | | |
| PM07 | Saccharomyces | — | — | — | — | — |
| | cerevisiae/BMH2 | | | | | |
| PM08 | Human/DLG4 | 0.3 | 0.3 | 1.2 | 0.4 | 0.4 |
| PM09 | Human/BRCA1 | 0.1 | 0.1 | — | — | — |
| | Tandem domain | | | | | |
| PM10 | Human/MCPH1 | 0.3 | 0.2 | 1.0 | 0.3 | 0.3 |
| PM11 | Human/TOPBP1 | 0.2 | 0.2 | — | — | — |
| PM12 | Mycobacterium | 0.7 | 0.6 | 7.1 | 6.9 | 0.6 |
| | tuberculosis/FhaA | | | | | |
| PM13 | Saccharomyces | 0.3 | 0.2 | 3.2 | 1.8 | 0.3 |
| | cerevisiae/FHA1 | | | | | |
| PM14 | Saccharomyces | 0.2 | 0.2 | 1.0 | 1.1 | 0.1 |
| | cerevisiae/RAD53 | | | | | |
| PM15 | Human/Pin1 | 0.9 | 0.5 | 7.4 | 0.9 | 0.4 |
| PM16 | Human/PLK1 | 0.6 | 0.5 | 2.1 | 1.7 | 0.5 |
| PM17 | Human/DIDO1 | 0.2 | 0.2 | — | — | — |
| PM18 | Human/MINT | 0.6 | 0.9 | 1.6 | 0.7 | 0.6 |
| PM19 | Human/RBM15 | 0.1 | 0.1 | 1.0 | 0.3 | 0.2 |
| PM20 | Human/RB15B | 0.2 | 0.2 | 0.5 | 0.3 | 0.2 |
| PM21 | Human/PHF3 | 0.2 | 0.1 | 0.5 | 0.3 | 0.2 |
| PM22 | Schizosaccharomyces | 0.1 | 0.2 | — | — | — |
| | pombe/FCP1 | | | | | |
| PM23 | Human/Scp1 | 0.1 | 0.1 | 0.3 | 0.2 | 0.2 |

pSA = pSAKLDEESILKQ-Biotin; SA = SAKLDEESILKQ-Biotin; pTQR = pTQRFLAYPDDD-Biotin; pTA = pTAKLDEESILKQ-Biotin; TA = TAKLDEESILKQ-Biotin.

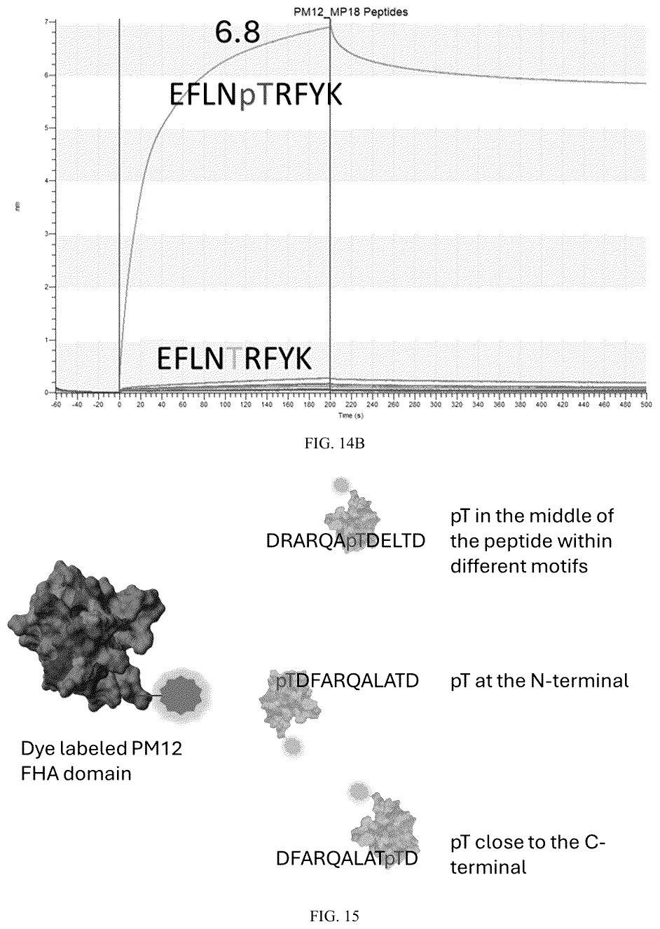

Next, control peptides (EFLNTRFY and TQRFLAYPDDD) were compared to model peptides (EFLN-pT-RFYK and pT-QRFLAYPDDD) to evaluate phospho-threonine detection in different peptide motifs using a post-translational modification-specific (PTM-specific) affinity reagent that binds to phospho-threonine (PM12). Using the Octet binding platform, Octet sensors were coated with the peptide of interest and dipped in buffer containing the PTM-specific affinity reagent. The results showed a positive shift (6.8 for EFLN-pT-RFYK; 4.4 for pT-QRFLAYPDDD) when the phospho-threonine was in the middle of the peptide or at the N-terminus of the peptide, indicating strong affinity of the PTM-specific affinity reagent to phospho-threonine in the peptides. In contrast, no shift was detected with the corresponding control peptides having unphosphorylated threonine (FIGS. 14A-14B).

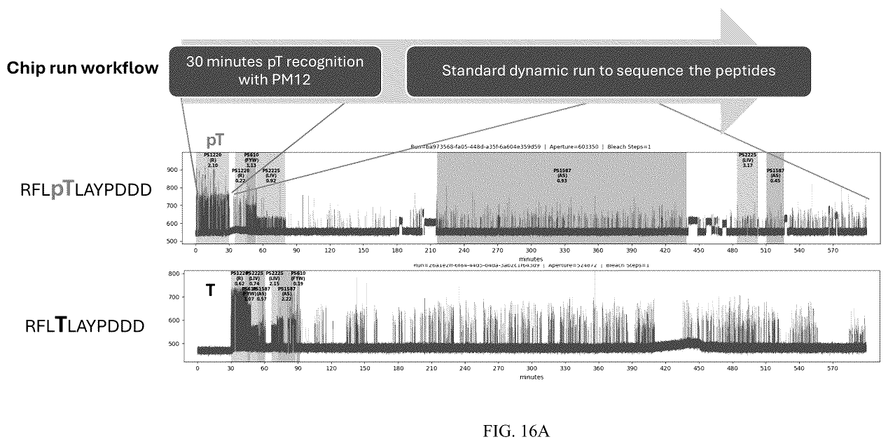

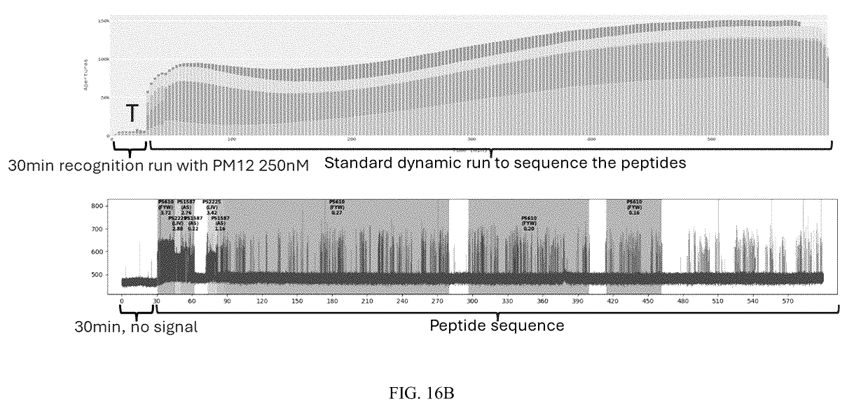

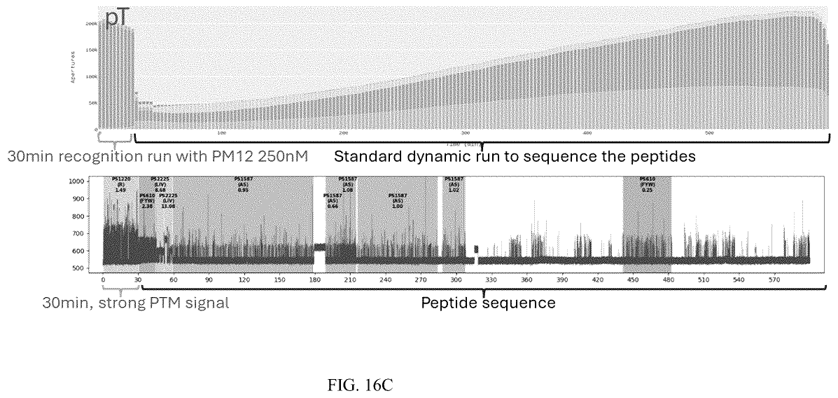

The phospho-threonine-specific affinity reagent, PM12, was evaluated for its ability to bind phospho-threonine residues residing in a range of motifs and at different positions in peptides (FIG. 15). To evaluate the PTM-specific affinity reagent, two-stage on-chip recognition runs were performed as described in Example 3. Using a model peptide (RFL-pT-LAYPDDD), fast pulsing derived from the interaction between the phospho-threonine and the PTM-specific affinity reagent was detected in the first 30 minutes. In contrast, no pulsing was detected in the first 30 minutes when using a control peptide that did not comprise a phospho-threonine (RFLTLAYPDDD). Peptide sequencing following PTM detection was not affected for either peptide (FIGS. 16A-16C).

The example results shown in FIGS. 16A-16C further demonstrated that two-stage recognition runs were successfully carried out in parallel at the single molecule level. As depicted by the workflow in FIG. 16A, the two-stage recognition runs included a first stage in which the PTM-specific affinity reagent was added to a chip to determine the presence or absence of phospho-threonine in single peptide molecules immobilized in individual apertures on the chip. This was followed by a second stage in which reagents for peptide sequencing (amino acid recognition molecules, cleaving reagents) were added to the chip to determine sequence information for the single peptide molecules. The plots shown at the top of FIGS. 16B and 16C are stacked plots showing the number of apertures (y-axis) with recognition taking place on the chip over the course of two-stage recognition runs (x-axis) performed in parallel with peptides having unphosphorylated threonine (FIG. 16B) or phosphorylated threonine (FIG. 16C). Each plot shown at the bottom of FIGS. 16B and 16C is an example binding trace from a single aperture on the chip. As shown in the bottom plot of FIG. 16C, both stages of the recognition run with phospho-threonine peptide displayed signal pulsing indicative of on/off binding events between the peptide and the reagents used for PTM detection and peptide sequencing.

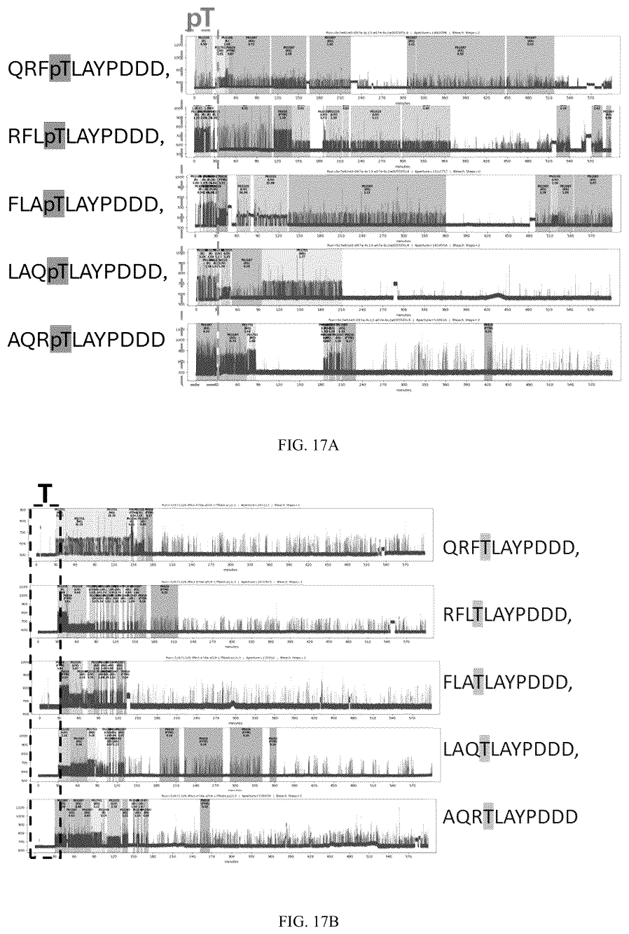

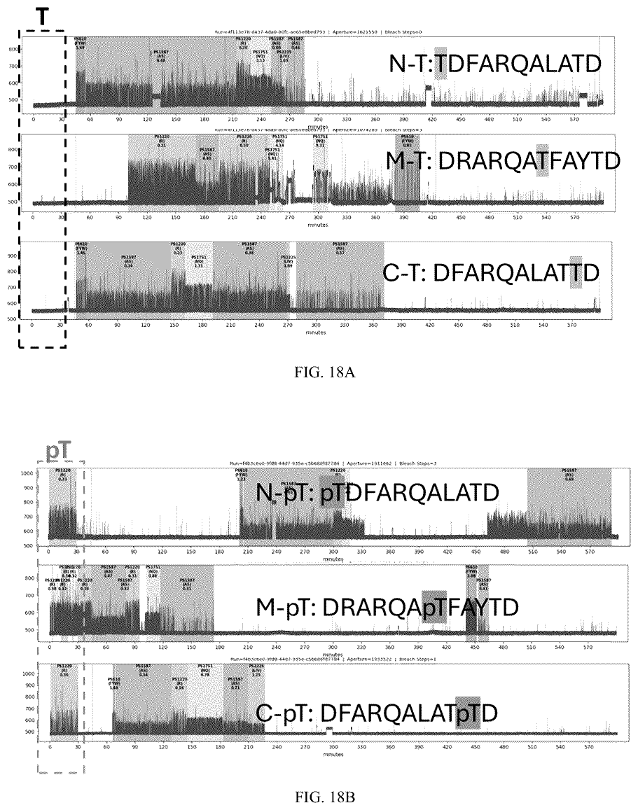

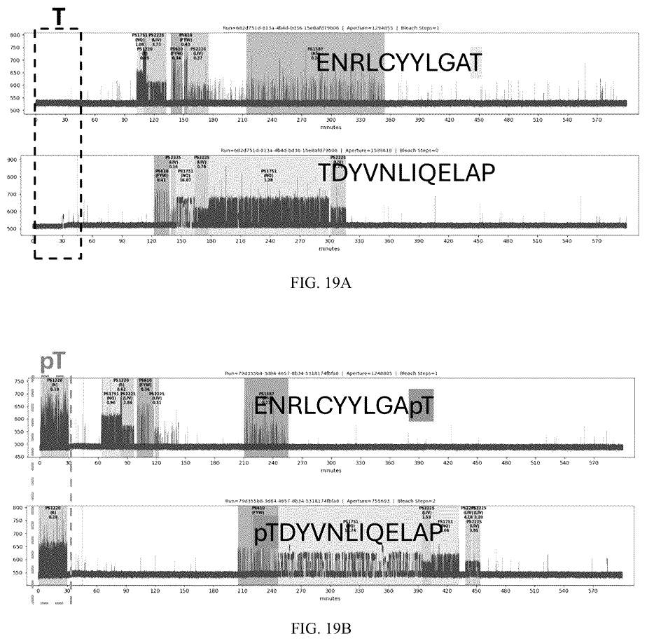

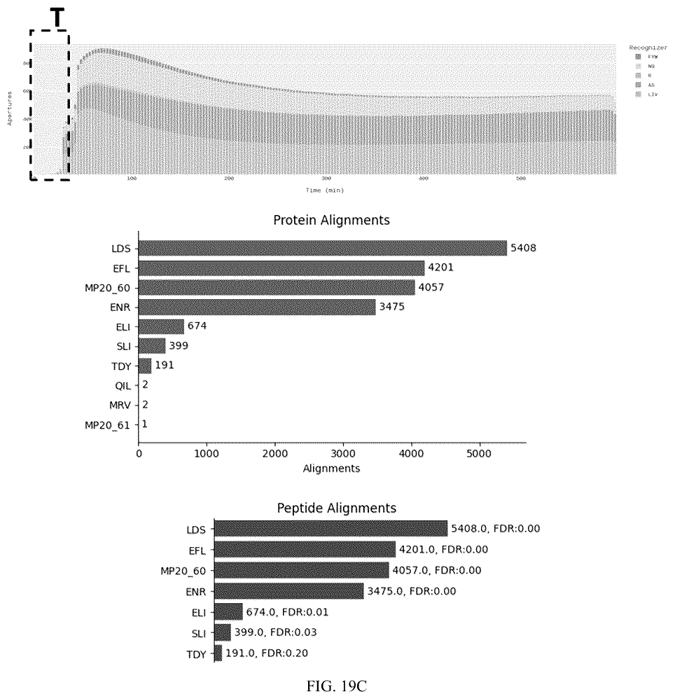

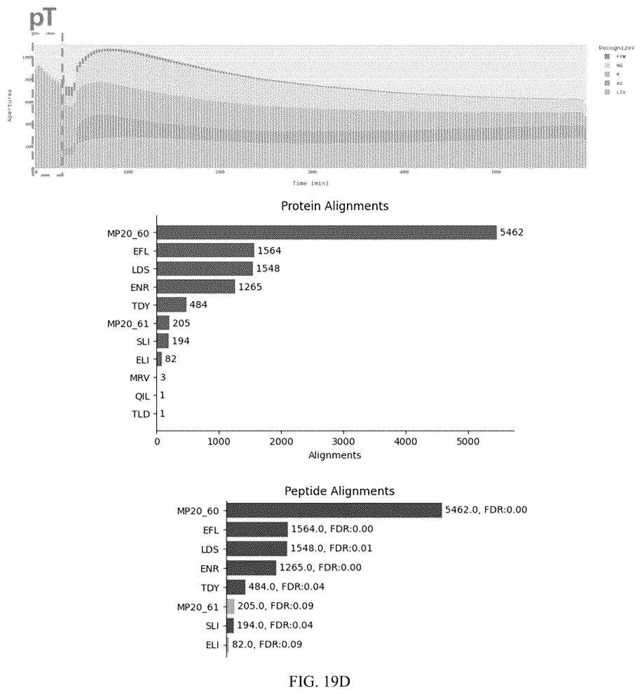

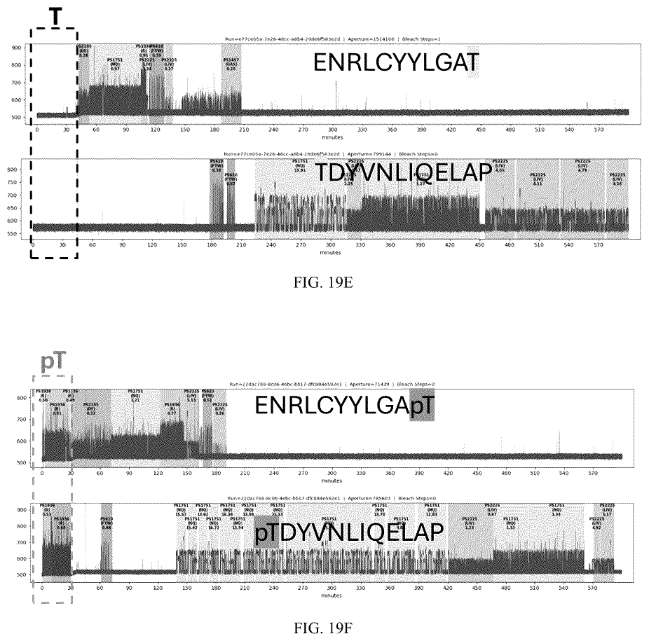

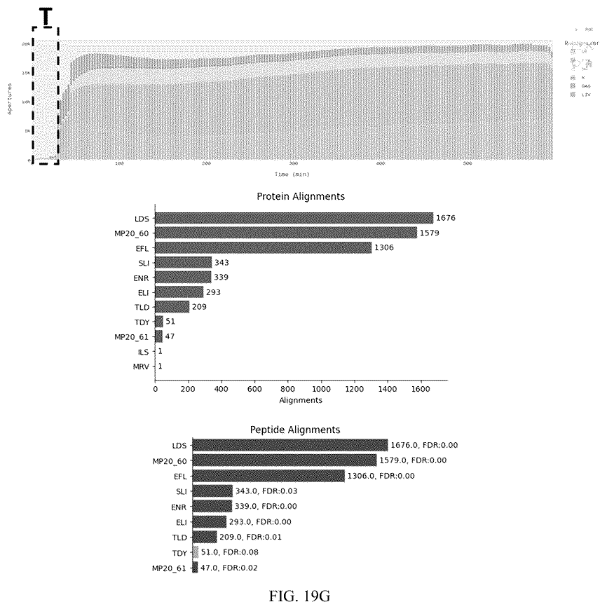

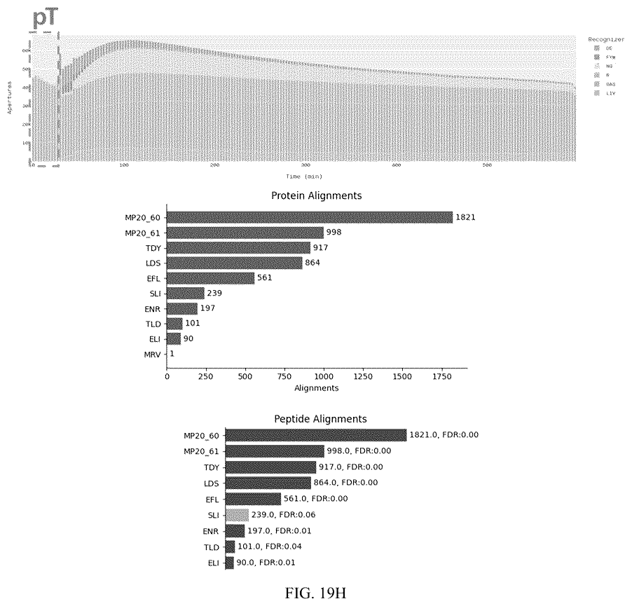

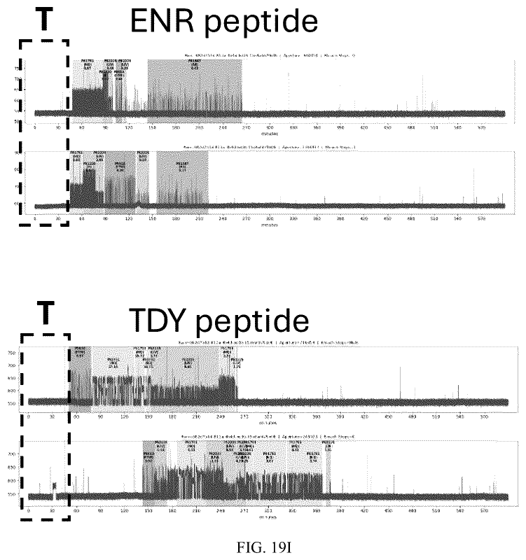

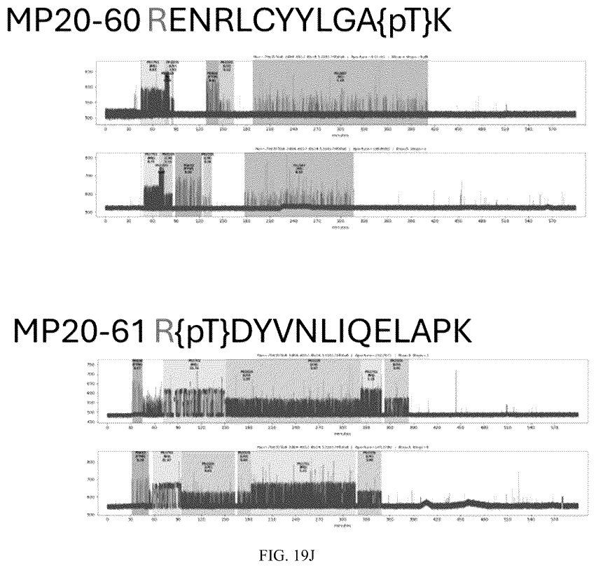

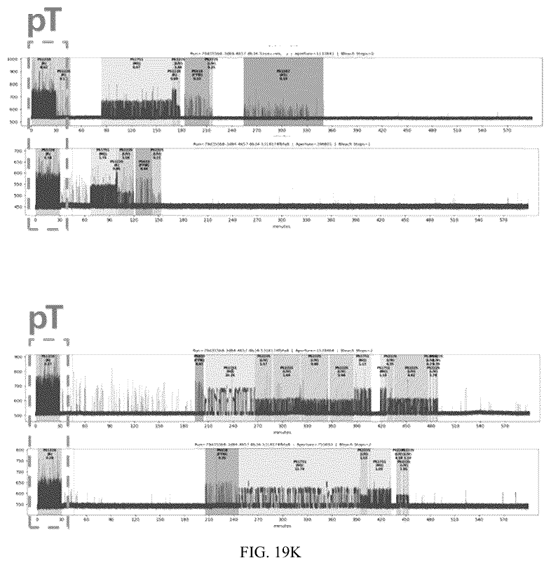

Using the two-stage recognition run format described above, PTM recognition of phospho-threonine in various peptide motifs was evaluated. Phospho-threonine detection by the PTM-specific affinity reagent in different peptide motifs was evaluated with unphosphorylated threonine (QRFTLAYPDDD, RFLTLAYPDDD, FLATLAYPDDD, LAQTLAYPDDD, and AQRTLAYPDDD) and compared to their phosphorylated threonine counterparts (QRF-pT-LAYPDDD, RFL-pT-LAYPDDD, FLA-pT-LAYPDDD, LAQ-PT-LAYPDDD, and AQR-pT-LAYPDDD). The PTM-specific affinity reagent was able to detect phospho-threonine in all five peptide motifs tested, with pulsing detected in the first 30 minutes of each run (FIG. 17A). In contrast, no signal was detected for any of the unphosphorylated peptide motifs (FIG. 17B). The PTM-specific affinity reagent was also evaluated for its ability to detect phospho-threonine residues at different positions in peptides. Pulsing in the first 30 minutes demonstrated that the PTM-specific affinity reagent recognized phospho-threonine residues at the N-terminus, the C-terminus, and in the middle of peptides, while no signal was observed for unphosphorylated threonine residues at corresponding positions in peptides (FIGS. 18A-18B). Similar results were obtained by performing the same two-stage chip run on phospho-threonine peptides spiked in the CDNF libraries (FIGS. 19A-19H). Analysis of two peptide trace showed that the PTM-specific affinity reagent is specific for phospho-threonine, binding to pT-ENR and pT-TDY peptides but not the ENR and TDY peptides in the CDNF library. Additionally, the PTM-specific affinity reagent was able to discriminate between phosphorylated and unphosphorylated CDNF peptides in the same run (FIGS. 19I-19K).

This Example demonstrates the use of a PTM-specific affinity reagent to identify phospho-threonine residues in different peptide motifs at the N-terminus, C-terminus, and middle of the peptide. This Example further demonstrates the use of a PTM-specific affinity reagent in a two stage chip run for sequential proteoform identification and peptide sequencing.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

What is claimed is:

1. A single-molecule measurement method comprising:
(a) contacting a single polypeptide with one or more post-translational modification-specific (PTM-specific) affinity reagents; and
(b) identifying whether the single polypeptide comprises a post-translational modification (PTM) by determining a luminescence signature representative of the binding interaction(s) between the single polypeptide and the one or more PTM-specific affinity reagents.

2. The method of claim 1, wherein the method further comprises:
(c) contacting the single polypeptide with one or more terminal amino acid recognition molecules; and
(d) detecting a series of signal pulses indicative of association of the one or more terminal amino acid recognition molecules with successive amino acids exposed at a terminus of the single polypeptide while the single polypeptide is being degraded.

3. The method of claim 1, wherein the single polypeptide is a full-length protein.

4. The method of claim 3, wherein the method further comprises:
contacting the full-length protein with one or more protein-specific affinity reagents, wherein each protein-specific affinity reagent binds to an amino acid that does not comprise a PTM; and
determining a further luminescence signature representative of the binding interaction(s) between the single polypeptide and the one or more protein-specific affinity reagents, wherein the further luminescence signature is further indicative of whether the single polypeptide comprises the PTM.

5. The method of claim 4, wherein the one or more protein-specific affinity reagents comprise antibodies or aptamers.

6. The method of any one of claims 3-5, wherein the method further comprises:
(c) fragmenting the full-length protein into a plurality of peptides;
(d) contacting one or more peptides of the plurality with one or more terminal amino acid recognition molecules; and
(e) detecting a series of signal pulses indicative of association of the one or more terminal amino acid recognition molecules with successive amino acids exposed at each terminus of the one or more peptides while the one or more peptides are being degraded.

7. A method of polypeptide sequencing comprising:
(a) contacting a chip array comprising a plurality of compartments with a plurality of polypeptides;
(b) immobilizing each polypeptide of the plurality of polypeptides to a surface of the chip array;
(c) contacting the plurality of polypeptides with one or more post-translational modification-specific (PTM-specific) affinity reagents; and
(d) identifying whether each polypeptide comprises a post-translational modification (PTM) by determining a luminescence signature representative of the binding interaction(s) between each polypeptide and the one or more PTM-specific affinity reagents.

8. The method of claim 7, wherein the method further comprises:
(e) contacting each polypeptide with one or more terminal amino acid recognition molecules; and
(f) detecting a series of signal pulses indicative of association of the one or more terminal amino acid recognition molecules with successive amino acids exposed at a terminus of each polypeptide while each polypeptide is being degraded, thereby sequencing each polypeptide.

9. The method of claim 7, wherein the plurality of polypeptides are full-length proteins.

10. The method of claim 9, wherein the method further comprises:
(e) fragmenting the full-length proteins into a plurality of peptides;
(f) contacting one or more peptides of the plurality with one or more terminal amino acid recognition molecules; and
(g) detecting a series of signal pulses indicative of association of the one or more terminal amino acid recognition molecules with successive amino acids exposed at each terminus of the one or more peptides while the one or more peptides are being degraded, thereby sequencing the one or more peptides.

11. A method of characterizing proteoforms of a polypeptide comprising:
(a) contacting a chip array comprising a plurality of compartments with a sample comprising a first proteoform of a polypeptide and a second proteoform of a polypeptide, wherein the post-translational modification (PTM) profile of the first proteoform is different than the PTM profile of the second proteoform;
(b) immobilizing the first proteoform to a surface of a first compartment of the chip array and the second proteoform to a surface of a second compartment of the chip array;
(c) contacting the first proteoform and the second proteoform with one or more post-translational modification-specific (PTM-specific) affinity reagents; and
(d) identifying whether the first proteoform and/or the second proteoform comprises a post-translational modification (PTM) by determining a luminescence signature representative of the binding interaction(s) between each proteoform and the one or more PTM-specific affinity reagents.

12. The method of claim 11, wherein the method further comprises:
(e) contacting the first proteoform and/or the second proteoform with one or more terminal amino acid recognition molecules; and
(f) detecting a series of signal pulses indicative of association of the one or more terminal amino acid recognition molecules with successive amino acids exposed at a terminus of each proteoform while each proteoform is being degraded, thereby sequencing the first proteoform and the second proteoform.

13. The method of claim 11, wherein the first and second proteoforms are full-length proteins.

14. The method of claim 13, wherein the method further comprises:
contacting the first and second proteoforms with one or more protein-specific affinity reagents, wherein each protein-specific affinity reagent binds to an amino acid that does not comprise a PTM; and
determining a further luminescence signature representative of the binding interaction(s) between the one or more protein-specific affinity reagents and the first proteoform and/or second proteoform, wherein the further luminescence signature is further indicative of whether the first proteoform and/or the second proteoform comprises a PTM.

15. The method of claim 14, wherein the one or more protein-specific affinity reagents comprise antibodies or aptamers.

16. The method of any one of claims 13-15, wherein the method further comprises:
(e) fragmenting the full-length proteins into a plurality of peptides;
(f) contacting one or more peptides of the plurality with one or more terminal amino acid recognition molecules; and
(g) detecting a series of signal pulses indicative of association of the one or more terminal amino acid recognition molecules with successive amino acids exposed at each terminus of the one or more peptides while the one or more peptides are being degraded, thereby sequencing the one or more peptides.

17. The method of any one of the preceding claims, wherein the one or more PTM-specific affinity reagents are antibodies or aptamers.

18. The method of any one of the preceding claims, wherein the one or more PTM-specific affinity reagents specifically bind to an amino acid comprising a phosphorylation, a glycosylation, acetylation, ADP-ribosylation, citrullination, formylation, N-linked glycosylation, O-linked glycosylation, hydroxylation, methylation, myristoylation, neddylation, nitration, oxidation, palmitoylation, prenylation, S-nitrosylation, sulfation, sumoylation, or ubiquitination.

19. The method of any one of the preceding claims, wherein the one or more PTM-specific affinity reagents specifically bind to phospho-tyrosine, phospho-serine, or phospho-threonine.

20. The method of any one of the preceding claims, wherein the one or more PTM-specific affinity reagents comprise one or more PTM-specific affinity reagents selected from Table 1.

21. The method of any one of the preceding claims, wherein the one or more PTM-specific affinity reagents comprise one or more PTM-specific affinity reagents selected from PM49, PS33, and PM12.

22. The method of any one of the preceding claims, wherein the one or more PTM-specific affinity reagents is labeled.

23. The method of claim 22, wherein the label is a luminescent label or a conductivity label.

24. The method of claim 23, wherein the luminescent label comprises at least one fluorophore dye molecule.

25. The method of claim 24, wherein the luminescent label comprises 20 or fewer fluorophore dye molecules.

26. The method of any one of the preceding claims, wherein the polypeptide(s) are contacted with two or more PTM-specific affinity reagents at the same time.

27. The method of claim 26, wherein each of the two or more PTM-specific affinity reagents comprise a unique label relative to the other PTM-specific affinity reagents.

28. The method of any one of the preceding claims, wherein the polypeptide(s) are contacted in series with a first PTM-specific affinity reagent and a second PTM-specific affinity reagent, optionally wherein the first PTM-specific affinity reagent is removed prior to addition of the second PTM-specific affinity reagent.

29. The method of any one of the preceding claims, wherein determining the luminescence signature comprises detecting a series of signal pulses indicative of association of the one or more PTM-specific affinity reagents with the PTM of the polypeptide(s).

30. The method of claim 29, wherein detecting a series of signal pulses indicative of association of the one or more PTM-specific affinity reagents with the PTM of the polypeptide(s) allows for a determination of the type of amino acids located at positions in proximity to the PTM of the polypeptide(s).

31. The method of claim 29, wherein detecting a series of signal pulses indicative of association of the one or more PTM-specific affinity reagents with the PTM of the polypeptide(s) allows for a determination of the location of the PTM within the polypeptide(s).

32. The method of claim 29, wherein detecting a series of signal pulses indicative of association of the one or more PTM-specific affinity reagents with the PTM of the polypeptide(s) assists with a determination of the amino acid sequence of the polypeptide(s).

33. The method of any one of the preceding claims, wherein the PTM is to an amino acid comprising a phosphorylation, a glycosylation, acetylation, ADP-ribosylation, citrullination, formylation, N-linked glycosylation, O-linked glycosylation, hydroxylation, methylation, myristoylation, neddylation, nitration, oxidation, palmitoylation, prenylation, S-nitrosylation, sulfation, sumoylation, or ubiquitination.

34. The method of any one of the preceding claims, wherein the PTM is phospho-tyrosine, phospho-serine, or phospho-threonine.

35. The method of any one of the preceding claims, wherein contacting the polypeptide(s) with one or more terminal amino acid recognition molecules further comprises contacting the polypeptide(s) with a cleaving reagent.

36. The method of claim 35, wherein the cleaving reagent is an aminopeptidase.

37. The method of any one of the preceding claims, wherein the method allows for identification of the presence of the PTM at any location in the polypeptide(s).

38. The method of any one of the preceding claims further comprising washing the polypeptide(s) after determining the luminescence signature of the polypeptide(s) in the presence of the one or more PTM-specific affinity reagents.

39. The method of any one of the preceding claims further comprising fragmenting the polypeptide(s) prior to step (a).

40. The method of claim 39, wherein the fragmenting comprises fragmenting by cleaving and/or digesting the polypeptide(s).

41. The method of any one of the preceding claims, wherein association of the one or more terminal amino acid recognition molecules with each type of amino acid exposed at the terminus produces a characteristic pattern in the series of signal pulses that is different from other types of amino acids exposed at the terminus, optionally wherein the characteristic pattern comprises a portion of the series of signal pulses.

42. The method of claim 41, wherein a signal pulse of the characteristic pattern corresponds to an individual association event between a terminal amino acid recognition molecule and an amino acid exposed at the terminus.

43. The method of claim 42, wherein the signal pulse of the characteristic pattern comprises a pulse duration that is characteristic of a dissociation rate of binding between the terminal amino acid recognition molecule and the amino acid exposed at the terminus.

44. The method of claim 43, wherein each signal pulse of the characteristic pattern is separated from another by an interpulse duration that is characteristic of an association rate of terminal amino acid recognition molecule binding.

45. The method of any one of claims 41-44, wherein the characteristic pattern corresponds to a series of reversible terminal amino acid recognition molecule binding interactions with the amino acid exposed at the terminus of the single polypeptide molecule.

46. The method of any one of claims 41-45, wherein the characteristic pattern is indicative of the amino acid exposed at the terminus of the single polypeptide molecule and an amino acid at a contiguous position.

47. A composition comprising three or more PTM-specific affinity reagents.

48. The composition of claim 47, wherein the three or more PTM-specific affinity reagents are antibodies or aptamers.

49. The composition of claim 47 or 48, wherein the three or more PTM-specific affinity reagents specifically bind to an amino acid comprising a phosphorylation, a glycosylation, acetylation, ADP-ribosylation, citrullination, formylation, N-linked glycosylation, O-linked glycosylation, hydroxylation, methylation, myristoylation, neddylation, nitration, oxidation, palmitoylation, prenylation, S-nitrosylation, sulfation, sumoylation, or ubiquitination.

50. The composition of any one of claims 47-49 comprising a PTM-specific affinity reagent that specifically binds to phospho-tyrosine, a PTM-specific affinity reagent that specifically binds to phospho-serine, and a PTM-specific affinity reagent that specifically binds to phospho-threonine.

51. The composition of any one of claims 47-50, wherein the one or more PTM-specific affinity reagents is labeled.

52. The composition of claim 51, wherein the label is a luminescent label or a conductivity label.

53. The composition of claim 52, wherein the luminescent label comprises at least one fluorophore dye molecule.

54. The composition of claim 53, wherein the luminescent label comprises 20 or fewer fluorophore dye molecules.

55. The composition of any one of claims 47-54, wherein each of the three or more PTM-specific affinity reagents comprise a unique label relative to the other PTM-specific affinity reagents.

56. The composition of any one of claims 47-55, further comprising a sample comprising polypeptides.

57. The composition of any one of claims 47-55, further comprising a cleaving reagent.

58. A method of polypeptide analysis, the method comprising:
a) contacting a polypeptide with a composition comprising one or more PTM-specific affinity reagents;
b) monitoring a signal for a first series of signal pulses corresponding to binding interactions between the one or more PTM-specific affinity reagents and the polypeptide;
c) contacting the polypeptide with a composition comprising one or more amino acid recognition molecules;
d) monitoring the signal for a second series of signal pulses corresponding to binding interactions between the one or more amino acid recognition molecules and the polypeptide; and
e) determining one or more chemical characteristics of the polypeptide based on one or more characteristic patterns in the first and/or second series of signal pulses.

59. The method of claim 58, wherein (e) comprises identifying at least one PTM in the polypeptide based on a characteristic pattern in the first series of signal pulses.

60. The method of claim 58 or 59, wherein the at least one PTM comprises phospho-threonine, phospho-serine, phospho-tyrosine, or any combination thereof.

61. The method of any one of claims 58-60, wherein (e) comprises identifying one or more types of amino acids in the polypeptide based on a characteristic pattern in the second series of signal pulses.

62. The method of any one of claims 58-61, further comprising washing the polypeptide after (b) and prior to (c).

63. The method of claim 62, wherein the washing comprises contacting the polypeptide with a wash solution after (b) and prior to (c).

64. The method of any one of claims 58-63, wherein the composition of (a) does not comprise a cleaving reagent.

65. The method of any one of claims 58-64, wherein the composition of (c) comprises one or more cleaving reagents.

66. The method of any one of claims 58-65, wherein the one or more PTM-specific affinity reagents comprise antibodies or aptamers.

67. The method of claim 66, wherein the one or more PTM-specific affinity reagents comprise antibodies.

68. The method of any one of claims 58-67, wherein the one or more amino acid recognition molecules of (c) do not comprise antibodies.

69. The method of any one of claim 58-68, wherein the polypeptide is a full-length protein.

70. The method of claim 69, wherein the method further comprises fragmenting the full-length protein into a plurality of peptides after (b) and prior to (c).

71. The method of any one of claims 58-70, further comprising repeating (a) and (b) one or more times prior to (c).

72. The method of claim 71, wherein the repeating comprises contacting the polypeptide with one or more PTM-specific affinity reagents that are different from the one or more PTM-specific affinity reagents in the composition of (a).

73. The method of claim 71 or 72, further comprising washing the polypeptide before the repeating.

74. The method of any one of claims 58-73, wherein the one or more PTM-specific affinity reagents are labeled.

75. The method of claim 74, wherein the label is a luminescent label or a conductivity label.

76. The method of any one of claims 58-75, wherein the one or more PTM-specific affinity reagents specifically bind to an amino acid comprising a PTM selected from phosphorylation, glycosylation, acetylation, ADP-ribosylation, citrullination, formylation, N-linked glycosylation, O-linked glycosylation, hydroxylation, methylation, myristoylation, neddylation, nitration, oxidation, palmitoylation, prenylation, S-nitrosylation, sulfation, sumoylation, and ubiquitination.

77. The method of any one of claims 58-76, wherein at least one PTM-specific affinity reagent specifically binds to phospho-tyrosine, phospho-serine, or phospho-threonine.

78. The method of any one of claims 58-77, wherein the one or more PTM-specific affinity reagents comprise one or more PTM-specific affinity reagents selected from Table 1.

79. The method of any one of claims 58-78, wherein the one or more PTM-specific affinity reagents comprise a PTM-specific affinity reagent that specifically binds to phospho-tyrosine.

80. The method of claim 79, wherein the PTM-specific affinity reagent is PM49.

81. The method of claim 80, wherein the PTM-specific affinity reagent that specifically binds to phospho-tyrosine comprises an SH2 domain.

82. The method of claim 81, wherein the PTM-specific affinity reagent is PS33.

83. The method of any one of claims 58-82, wherein the one or more PTM-specific affinity reagents comprise a PTM-specific affinity reagent that specifically binds to phospho-threonine.

84. The method of claim 83, wherein the PTM-specific affinity reagent that specifically binds to phospho-threonine comprises an FHA domain.

85. The method of claim 84, wherein the PTM-specific affinity reagent is PM12.

86. The method of any one of claims 58-85, wherein the polypeptide is immobilized to a surface of a substrate comprising an array of compartments.

87. The method of claim 86, wherein the polypeptide is immobilized within a first compartment of the array.

88. The method of claim 87, wherein a different proteoform of the polypeptide is immobilized within a second compartment of the array.

89. The method of claim 88, wherein the method comprises performing (a) to (d) for each proteoform of the polypeptide, wherein (e) comprises identifying each proteoform of the polypeptide based on differences in the one or more chemical characteristics.

90. A method of polypeptide analysis, the method comprising:
a) contacting a substrate comprising an array of compartments with one or more PTM-specific affinity reagents, wherein each of at least two compartments of the array comprises a different proteoform of a polypeptide;
b) detecting a first series of signal pulses corresponding to binding interactions between the one or more PTM-specific affinity reagents and a first proteoform of the polypeptide in a first compartment;
c) detecting a second series of signal pulses corresponding to binding interactions between the one or more PTM-specific affinity reagents and a second proteoform of the polypeptide in a second compartment; and
d) identifying one or more PTMs in each of the first and second proteoforms based on one or more characteristic patterns in each of the respective first and second series of signal pulses.

91. The method of claim 90, further comprising sequencing the first and second proteoforms of the polypeptide.

92. The method of claim 91, wherein the sequencing comprises identifying one or more types of amino acids in each of the first and second proteoforms of the polypeptide.

93. The method of claim 91 or 92, wherein the sequencing comprises:
e) contacting the substrate with a composition comprising one or more amino acid recognition molecules; and
f) detecting a series of signal pulses corresponding to binding interactions between the one or more amino acid recognition molecules and successive amino acids exposed at a terminus of each proteoform while each proteoform is being degraded, thereby sequencing the first and second proteoforms.

94. The method of claim 90, wherein the first and second proteoforms are full-length proteins.

95. The method of claim 94, wherein the method further comprises:
fragmenting the full-length proteins into a plurality of peptides;
contacting one or more peptides of the plurality with one or more terminal amino acid recognition molecules; and
detecting a series of signal pulses indicative of association of the one or more terminal amino acid recognition molecules with successive amino acids exposed at each terminus of the one or more peptides while the one or more peptides are being degraded, thereby sequencing the one or more peptides.

96. The method of any one of claims 90-95, wherein (d) comprises determining that the first and second proteoforms are different proteoforms of the polypeptide.

97. The method of any one of claims 90-96, wherein the first proteoform of the polypeptide is immobilized to a surface of the first compartment, and wherein the second proteoform of the polypeptide is immobilized to a surface of the second compartment.
